(19) United States
(12) Patent Application Publication
(10) Pub. No.: US 2023/0372335 A1
(43) Pub. Date: Nov. 23, 2023

(54) TREATMENT OF COGNITIVE IMPAIRMENT WITH A CNS-PENETRANT sGC STIMULATOR

(71) Applicant: Cyclerion Therapeutics, Inc., Cambridge, MA (US)

(72) Inventors: Christopher John Winrow, Sudbury, MA (US); Christopher Ian Wright, Cambridge, MA (US); Chad Edward Glasser, Somerville, MA (US); Phillip M. Alday, Belle Chasse, LA (US); Alexander Ross Arslan, Redmond, WA (US); David F. Kleinschmidt, Princeton, NJ (US); Jacob Donoghue, Boston, MA (US)

(73) Assignee: Cyclerion Therapeutics, Inc., Cambridge, MA (US)

(21) Appl. No.: 18/031,371

(22) PCT Filed: Oct. 12, 2021
(86) PCT No.: PCT/US2021/054632

§ 371 Date: Apr. 12, 2023

Related U.S. Application Data
(60) Provisional application No. 63/135,797, filed on Jan. 11, 2021.
(60) Provisional application No. 63/090,849, filed on Oct. 13, 2020.

(51) Int. Cl. A61K 31/4985; A61K 9/00; A61K 45/06; A61P 25/28

(52) U.S. Cl. CPC A61K 31/4985; A61K 9/0053; A61K 45/06; A61P 25/28

(57) ABSTRACT

The present invention relates to a method of treating cognitive impairment in a patient in need thereof by administering Compound (I), a stimulator of soluble guanylate cyclase (sGC) at certain dosages either alone or in combination therapy.

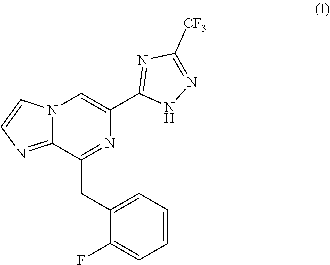 [SMILES: Fc1ccccc1Cc1nc(-c2nc(C(F)(F)F)n[nH]2)cn2ccnc12]

### RELATED APPLICATIONS

This application claims the benefit of the filing date, under 35 U.S.C. § 119(e), of U.S. Provisional Application No. 63/090,849, filed on Oct. 13, 2020 and U.S. Provisional Application No. 63/135,797, filed on Jan. 11, 2021. The entire contents of each of the above-referenced applications are incorporated herein by reference.

### FIELD OF THE INVENTION

The present invention provides methods of treating cognitive impairment in human subjects in need of the treatment by administering specific dosage regimens of a CNS-penetrant stimulator of soluble guanylate cyclase (sGC) either alone or in combination therapy.

### BACKGROUND OF THE INVENTION

### Cognition and Cognitive Impairment or Decline

Cognitive impairment and cognitive decline are broad terms describing deficits in one or more higher brain functions that generally involve aspects of thinking and information processing (i.e., cognition). Included among these functions or aspects of cognition are perception, learning, memory (short- and long-term), attention, attentional control, focus, concentration, language production and comprehension, decision-making, problem-solving, reasoning, planning, reaction time to a stimulus, cognitive speed and capacity, cognitive processing, and visuospacial skills. Cognitive impairment can start suddenly or gradually and can be temporary or more permanent. It may also manifest short term, or may progress and worsen in a progressive manner, and this will depend on the underlying cause or causes. Some common causes of cognitive impairment may include medication side effects, metabolic imbalances, hormonal problems, vitamin or nutrient deficiencies, delirium, psychiatric illness, damage to brain neurons due to an injury (for example in stroke or other cerebral vessel diseases or due to a traumatic brain injury), neurodegenerative conditions or diseases, neuropsychiatric diseases, toxins, or viral or bacterial infections.

Clinically, cognitive impairment can range from mild cognitive impairment (MCI) to severe dementia, such as in later stages of Alzheimer's disease (AD). Cognitive impairment is a large and growing health problem in developed and developing countries. For example, AD, the most commonly diagnosed cause of dementia, affects about 5.5 million Americans. Globally, the World Health Organization estimates the number of people living with various forms of dementia is 35.6 million. The overall number is expected to double by the year 2030 and to triple by 2050. The impact of the disease on patients, caregivers, families and societies is physically, psychologically, and economically significant.

Without reaching the level of clinically defined cognitive impairment in the form of MCI or dementia, cognitive function may also be suboptimal under certain circumstances. This type of cognitive impairment is termed subclinical cognitive impairment or decline. For example, cognitive decline due to aging (i.e., cognitive aging) is well characterized. Given the increased cognitive demands of today's society, due to increased flow of information via multiple types of media, subclinical cognitive impairment may also heavily affect the daily life and the quality of life of individuals suffering from it (Mattson et al., Physiol Rev, 2000 Vol. 82, pp 637-672).

It is also possible for some people to experience subjective cognitive decline (SCD), characterized by self-experienced persistent cognitive decline in comparison with previous normal status but without impairment as measured by standardized cognition tests.

Dementia, MCI, subclinical cognitive impairment, cognitive aging, and SCD, are general terms describing the sequelae resulting from neurodegeneration or acute neuronal damage. In neurodegeneration, progressive loss of neurons and synapses takes place. Neuronal damage can also occur as a result of an acute event, such as in traumatic brain injury or as a result of an infection or exposure to toxins. Some diseases involve specific brain regions or neural pathways. Neurodegeneration and neuronal damage may be characterized for example, by measuring loss of neurological function or diminished brain performance (e.g., memory loss, language effects, executive function loss, reduction in attention, distractibility, short attention span, decreases in saccadic eye velocity and increases in saccadic eye latency, reaction time to a stimulus, etc.) or by measuring brain activity or neurophysiology through techniques such as electroencephalography (EEG), including quantitative electroencephalography (qEEG), or other physiological changes such as changes in the levels of certain biomarkers that are indicative of brain damage or neuroinflammation, etc; it can also be characterized using imaging techniques, by observing loss of neuronal tissue accompanied by specific histopathological findings such as β-amyloid plaques in AD, neurofibrillary tangles consisting of phosphorylated tau proteins, Lewy Bodies, atrophy, ischemia, infarcts, and sclerosis in other diseases. The causes of neurodegeneration are still not well understood.

Results of many preclinical studies in the field of cognition have not translated into useful human treatments so far. The currently available treatment options remain extremely limited. Therefore, there is an urgent need to develop novel therapeutic interventions that attenuate cognitive decline, or preserve a patient's current cognitive capacities, or improve cognitive capacities in said patients.

### SUMMARY OF THE INVENTION

In a first aspect of the invention, disclosed herein is a method of treating cognitive impairment in a patient in need thereof by administering a total oral daily dose of Compound I of between 10 mg and 15 mg or an equal quantity in moles of a pharmaceutically acceptable salt of Compound Ito said patient.

In a second aspect of the invention, disclosed herein is a Compound I or a pharmaceutically acceptable salt thereof for use in treating cognitive impairment in a patient in need thereof by administering a total oral daily dose of Compound I of between 10 mg and 15 mg or an equal quantity in moles of a pharmaceutically acceptable salt of Compound I to said patient.

In a third aspect of the invention, disclosed herein is the use of Compound I or a pharmaceutically acceptable salt thereof for the manufacture of a medicament for treating cognitive impairment in a patient in need thereof.

In a fourth aspect, the methods and uses of the invention do not result in a significant incidence of adverse events (AEs) associated with symptomatic hypotension.

In a fifth aspect, the methods and uses of the invention involve treatment in combination with one or more additional therapeutic agent.

### BRIEF DESCRIPTION OF THE FIGURES

FIG. 1 shows the mean impact on alpha band power in EEG recordings of subjects when treated with Compound I vs subjects when treated with placebo at different timepoints.

FIG. 2 shows the mean change from baseline in alpha band power in EEG recordings of anterior and posterior brain regions of subjects when treated with Compound I vs subjects when treated with placebo on Day 15 of the treatment.

FIG. 3 is an idealized plot of N200 amplitude vs age and shows that larger N200 negative amplitudes were observed with increasing age following treatment with Compound I as compared to treatment with placebo or pre-treatment values.

FIG. 4 is an idealized plot of N200 latency vs age and shows that smaller latency increases with increasing age were observed following treatment with Compound I as compared to treatment with placebo or pre-treatment values.

FIG. 5 shows least squares mean change from baseline with 95% confidence intervals for change from baseline in saccadic peak velocity in subjects when treated with Compound I and in subjects when treated with placebo by study day/timepoint.

FIG. 6 shows box-and-whisker-plots as well as individual averages of change from baseline on day 15 for saccadic peak velocity in subjects when treated with Compound I and in subjects when treated with placebo. The boxes represent the interquartile range (Q1-Q3, IQR), the line within represents the median, the whisker lines represent minimum and maximum observed values within 1.5×IQR; each circle represents an individual subject average change from baseline value for day 15. Mean saccadic peak velocity change from baseline is the average for each subject and for each treatment on day 15 post-dose.

FIG. 7 shows box-and-whisker-plots as well as individual average changes from baseline on day 15 for saccadic reaction time (i.e., latency) in subjects when treated with Compound I and in subjects when treated with placebo. The boxes represent the IQR (Q1-Q3); the line within represents the median; the whiskers lines represent the minimum and maximum observed values within 1.5×IQR; each circle represents an individual subject average change from baseline for day 15. Mean saccadic reaction time (latency) change from baseline is the average for each subject and for each treatment on day 15 post-dose.

FIG. 8 shows mean change versus placebo in MMN(N200) latency and how it is driven by the response in older subjects. The latency response was greater in subjects older than 70 years old than in subjects between 65 and 69 years old. The narrowing of variance for older subjects also supports a drug effect.

FIG. 9. shows least square mean concentration changes from placebo at day 15 for a number of biomarkers measured in the CSF of study subjects (nominal cutoff value of p less than 0.2).

### DETAILED DESCRIPTION OF THE INVENTION

### Definitions and General Terminology

Unless otherwise defined, all technical and scientific terms used herein have the same meaning as commonly understood by one of ordinary skill in the art to which this invention belongs, which is the field of medicine, and of brain medicine in particular. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the word “a” before a noun represents one or more of the particular noun. As used herein, the singular forms “a,” “an,” and “the” include plural referents unless the context clearly dictates otherwise.

As used herein, the terms “subject” and “patient” are used interchangeably. A subject or a patient is a human patient or human subject.

For the terms “for example” and “such as,” and grammatical equivalences thereof, the phrase “without limitation” or “and without limitation” is understood to follow unless explicitly stated otherwise.

Cognitive function naturally declines with age and also in pathological situations. “Cognitive impairment” refers to deficits in one or more higher brain functions that generally involve aspects of thinking and information processing (i.e., cognition). In some embodiments, cognitive impairment is manifested as reduced attention, short attention span, distractibility, reduced focus or reduced processing speed. As used herein, cognitive impairment includes mild cognitive impairment (MCI), dementia, subclinical cognitive impairment, subjective cognitive decline (SCD) and cognitive aging.

“Mild cognitive impairment (MCI)” is widely regarded in the field as the intermediate stage of cognitive impairment between the cognitive changes seen in normal cognitive aging and those associated with dementia. Elderly patients with MCI constitute a high-risk population for developing dementia, in particular Alzheimer's disease (AD).

Although patients with MCI may represent an optimal target population for pharmacological and non-pharmacological interventions, results from clinical trials have been mixed and an effective treatment remains elusive. Similarly, effective treatments for dementia (for example, in AD dementia) are still not available, despite the very large number of clinical trials conducted to date.

Although dementia and MCI are both common, even those who do not experience these conditions may experience subtle cognitive changes (“subclinical cognitive impairment or decline”). In some embodiment of the invention, this may be the result of aging (i.e., cognitive aging).

“Cognitive aging” is cognitive change as a normal process of aging and has been well documented in the scientific literature. Some cognitive abilities, such as vocabulary, are resilient to brain aging and may even improve with age. Other abilities, such as attention, focus, reaction time, executive function, conceptual reasoning, memory, processing speed, and psychomotor ability decline gradually over time after the third decade of life. By definition, normal age-related cognitive change does not impair a person's ability to perform basic daily activities. However, studies show that normal cognitive aging can result in subtle declines in complex functional performance or functional abilities, such as the ability to drive or to perform in certain professions.

“Dementia” is not a specific disease but is rather a general term for the impaired ability to perform certain cognitive functions such as paying attention, focusing, remembering, thinking, or making decisions, that interferes with carrying out everyday activities. Just like many different diseases or. conditions or events may cause subclinical or subjective clinical impairment or MCI, many diseases, conditions or events may cause dementia. AD is the most common cause of progressive dementia in older adults. Depending on the cause, some dementia symptoms may be reversible.

“Subjective cognitive decline (SCD)” is characterized by the patient's self-experienced persistent decline in cognitive capacity in comparison with a previously normal status that is unrelated to an acute event (Jessen et al., Alz Dem, 2014, Vol. 10, pp 844-852). There is increasing evidence that SCD in individuals with unimpaired performance on cognitive tests may represent the first symptomatic manifestation of AD or dementia. According to Jessen et al. some criteria for determining if a patient has SCD are: 1) being referred to a clinician for evaluation of cognitive complaints; 2) self-experienced persistent decline in cognitive capacity in comparison with a previously normal status that is unrelated to an acute event and 3) normal age-, gender-, and education- adjusted performance on standardized cognitive tests.

“Functional capacity” refers to a person's capability or ability to perform tasks and activities that people find necessary or desirable in their lives in different circumstances or situations. Functional capacity is most appropriately examined with reference to particular life-cycle tasks that an individual may need to perform. For instance, for children, functional requirements include learning at school, participating in play, and involvement in family life. For adults, functional abilities in the labor force are important, as well as activities related to rearing and interacting with their children. There are many tools known in the field that examine the ability to perform certain age-related tasks in detail. Other assessment tools, especially those used in large-scale research, attempt to use questions that work for all age groups. The most common approach is to consider ability to perform each in a list of specific tasks or activities that are most relevant to the population being studied. Some assessments of functional capacity focus, for instance, on activities related to cardiovascular capacity, or activities related to muscle strength or balance, or activities related to exercise capacity, or cognitive abilities. In the last few years, functional capacity measures have particularly been emphasized for people who need long-term care, including elderly people, but also younger people suffering from certain chronic diseases or disability. With reference to those needing long-term care, two common terms have emerged to characterize functional capacity: ability to perform “activities of daily living” (ADLs) and ability to perform “instrumental activities of daily living” (IADLs) (see for example Lara-Ruiz J, Kauzor K, Nakhala M, et al. The Functional Ability of MCI and Alzheimer's Patients Predicts Caregiver Burden. GeroPsych (Bern). 2019; 32(1):31-39 and references cited therein; and https://www.acc.org/latest-in-cardiology/articles/2018/07/10/07/16/prioritizing-the-importance-of-functional-capacity-assessments-among-the-older-population). Reduced functional capacity is also associated with reduced quality of life (QOL) and increased caretaker burden. Both QOL and caretaker burden can also be measured and tools for their assessment and quantification have been developed in the field.

ADLs are the most basic of self-care functions. These include things like bathing, dressing, using the toilet, transferring in and out of beds or chairs, and eating. When ADL is measured dichotomously, people are usually considered independent if they can do the function without help (even if they depend on equipment) and dependent if they need human help. Depending on the level of detail sought, some ADL measures use more graduated scales to measure degrees of dependency; some break down the tasks (e.g., dressing can include upper body, lower body, putting on shoes); and some add some quantitative measures (e.g., walking a certain number of feet, climbing a certain number of stairs).

IADLs are functions that may be needed for independence depending on task allocation in a family unit or demands made specific to a person's life or age. They may include things like cooking, cleaning, laundry, shopping, making and receiving telephone calls, driving or using public transportation, taking medicines or being able to perform certain work-related tasks.

Functional capacity can be measured by questions about what a person can do, or by demonstrations of actual ability (e.g., getting up from a chair, demonstrating ability to hold food on a spoon and bring it to one's mouth, opening a medicine bottle and taking out the correct number of pills, or carrying out more complex tasks). It can also be measured by questions about what a person actually does do (sometimes these are addressed by caretakers rather than patient him or herself). The measurement strategy should be tailored to aspect of functional capacity one intends to measure (Applegate, W. B.; Blass, J. P.; and Williams, T. E. (1990), Instruments for the Functional Assessment of Older Patients. New England Journal of Medicine 322(17): 1132-1148; Kane, R. L., and Kane, R. A. (2000), Assessing Older Persons: Measurement, Meaning, and Practical Applications. New York: Oxford University Press; McDowell, I.; and Newell, C. (1996), Measuring Health: A Guide to Rating Scales and Questionnaires, 2nd edition. New York: Oxford University Press).

Lack of functional capacity in general, and in ADL or IADL tasks in particular, can result from any combination of: physical problems, lack of social resources, lack of motivation (e.g., because of depression) and, most importantly for the purposes of this disclosure they can be the result of cognitive impairment. Decline or negative changes in cognitive parameters have been shown to be associated with diminished functional capacity as well as QOL. Conversely, improvements or positive changes in cognitive parameters have been shown to be positively linked to improvements in functional capacity and QOL in many patients.

The relationship between cognitive status and functional abilities or functional capacity has been examined in diverse patient populations with different levels of clinical or subclinical cognitive impairment. In general, it has been observed that cognitive impairment appears to be positively correlated with reductions in functional performance or functional capabilities and that better cognition is associated with higher functional capacity (see for example: Jing Ee Tan, David F. Hultsch & Esther Strauss, Cognitive abilities and functional capacity in older adults: results from the modified Scales of Independent Behavior—Revised. The Clinical Neuropsychologist, 23:3, 479-500, 2009; McClure M M, Harvey P D, Bowie C R, Iacoviello B, Siever L J, Functional outcomes, functional capacity, and cognitive impairment in schizotypal personality disorder. Schizophr Res. 2013; 144(1-3):146-150; McLennan S N, Mathias J L, Brennan L C, Russell M E, Stewart S. Cognitive impairment predicts functional capacity in dementia free patients with cardiovascular disease. J Cardiovasc Nurs. 2010; 25(5):390-397; Zielonka D, Ren M, De Michele G, et al. The contribution of gender differences in motor, behavioral and cognitive features to functional capacity, independence and quality of life in patients with Huntington's disease. Parkinsonism Relat Disord. 2018; 49:42-47; Ott C, Miné H, Petersen J Z, Miskowiak K. Relation between functional and cognitive impairments in remitted patients with bipolar disorder and suggestions for trials targeting cognition: An exploratory study. J Affect Disord. 2019; 257:382-389; Clark J M R, Jak A J, Twamley E W. Cognition and functional capacity following traumatic brain injury in veterans. Rehabil Psychol. 2020; 65(1):72-79).

Some approaches that improve brain health or performance in general, and cognition in particular, have been shown to also result in improvements in functional capacity (see, for example, Bherer L, Cognitive plasticity in older adults: effects of cognitive training and physical exercise. Ann N Y Acad Sci. 2015; 1337:1-6; Holzapfel S D, Ringenbach S D, Mulvey G M, et al., Improvements in manual dexterity relate to improvements in cognitive planning after assisted cycling therapy (ACT) in adolescents with down syndrome. Res Dev Disabil. 2015; 45-46:261-270; Winblad B, Kilander L, Eriksson S, et al., Donepezil in patients with severe Alzheimer's disease: double-blind, parallel-group, placebo-controlled study [published correction appears in Lancet. 2006 Jun. 17; 367(9527):1980] [published correction appears in Lancet. 2006 Nov. 11; 368(9548):1650]. Lancet. 2006; 367(9516):1057-1065.

Therefore, a pharmacological approach that improves the neurophysiology of the brain or improves measures of brain performance that are known to be related to one or more aspects of cognition would be expected to also result in an improvement in overall functional capacity in patients.

For example, a pharmacological approach that improves measures of brain performance related to aspects of cognition such as attention, focus, reaction time to a stimulus, or processing speed, may be useful in patients with reduced functional capacity.

The assessment of cognitive function and functional capacity and the corresponding pathology underlying the observed dysfunction, decline, or symptoms, may be carried out using a number of different assessment tools or clinical measurements known and used in the field. These range from imaging tools (e.g., MRIs, PET, CT scans), to laboratory measurements (e.g., fluid biomarkers measured in blood, cerebro-spinal-fluid or CSF, urine, plasma, serum, skin, saliva), to clinical outcome assessments (e.g., patient- or clinician-reported outcome instruments, performance outcome measures such as saccadic eye movement (SEM) assessments), digital assessments (e.g., wearable devices, sensor- or camera-based asessments) and others (e.g., EEG). Some of these are described in the Experimental section. Others are known in the art and could be used in the hospital or clinical setting. For example, the American Association of Family Physicians (AAFP), in its webpage, describes and provides links to a number of potential cognitive assessment tools, such as MiniCog, MoCA, SLUMS Examination, CPCoG, MIS and MMSE and others (https://www.aafp.org/patient-care/public-health/cognitive-care/cognitive-evaluation.html). Some measurements are carried out to help in diagnosis. Others are carried out to help in assessing prognosis. Others may be carried out to assess pharmacological responses to a certain intervention (pharmacodynamic assessments) such as described herein. Others may be carried out to assess susceptibility to or risk of cognitive or functional decline (e.g., assessment of genetic markers) or to assess cognitive impairment progression in a patient.

Attention or focus are important aspects of cognition and are defined as the ability to focus selectively on a selected stimulus, differentiate distractive stimuli, sustaining that focus and shifting it at will, or the ability to concentrate. Discrimination between stimuli is also an important component of cognitive performance, along with reaction time to a stimulus and information processing speed, enabling, as an example, a physical response to a target stimulus (see for example: McDonough I M, Wood M M, Miller W S Jr., A Review on the Trajectory of Attentional Mechanisms in Aging and the Alzheimer's Disease Continuum through the Attention Network Test. Yale J Biol Med. 2019 Mar. 25; 92(1):37-51. PMID: 30923472; Malhotra PA, Impairments of attention in Alzheimer's disease. Curr Opin Psychol. 2019 October; 29:41-48). Processing speed or reaction time in the presence of a stimulus has been used to distinguish between people with different levels of cognitive capacities, including in patients with AD or vascular disease (see for example Lu H, Chan S S M, Lam L C W, ‘Two-level’ measurements of processing speed as cognitive markers in the differential diagnosis of DSM-5 mild neurocognitive disorders (NCD). Sci Rep. 2017 Mar. 31; 7(1):521). For example, early responses to donepezil, a drug used for the treatment of AD, were assessed using measures of attention in a clinical trial (Vila-Castelar C, Ly J J, Kaplan L, Van Dyk K, Berger J T, Macina L O, Stewart J L, Foldi N S. Attention Measures of Accuracy, Variability, and Fatigue Detect Early Response to Donepezil in Alzheimer's Disease: A Randomized, Double-blind, Placebo-Controlled Pilot Trial. Arch Clin Neuropsychol. 2019 May 1; 34(3):277-289).

The term “therapeutically effective amount” or “pharmaceutically effective amount” as used herein means that amount of active compound or pharmaceutical agent that elicits the medicinal response in a human that is being sought by a medical doctor or other clinician. The therapeutically or pharmaceutically effective amount of a compound is at least the minimum amount necessary to ameliorate, palliate, lessen, delay, reduce, alleviate, or cure a disease, disorder, or syndrome or one or more of its symptoms, signs or causes. In another embodiment, it is the amount needed to bring abnormal levels of certain clinical markers of the disease, disorder, or syndrome closer to the normal values or levels. In another embodiment, it is the amount needed to bring the levels of certain clinical markers displayed by a subject closer to those of a normal subject of the same age (normalization) or closer to those of a younger subject. An effective amount can be administered in one or more administrations throughout the day.

As used herein, a dose that does not “result in a significant incidence of adverse events (AEs) associated with symptomatic hypotension” is one that does not result in excessive orthostatic hypotension, excessive dizziness, excessive postural dizziness, excessive pre-syncope, or excessive syncope in patients. Excessive orthostatic hypotension, excessive dizziness, excessive postural dizziness, excessive pre-syncope, or excessive syncope in patients are those that would warrant discontinuation of treatment by the patient or a recommendation of discontinuation by the practitioner.

The terms “administer”, “administering” or “administration” in reference to a compound or pharmaceutical agent, mean introducing the compound into the body of the patient in need of treatment. When Compound I or a pharmaceutically acceptable salt thereof is used in combination with one or more other therapeutic agents, “administration” and its variants are each understood to encompass concurrent and/or sequential introduction of Compound I and the other therapeutic agents into the patient.

“Treat”, “treating” or “treatment” with regard to a disorder, disease, condition, symptom or syndrome, refers to abrogating or improving the cause and/or the effects (i.e., the symptoms, physiological, physical, psychological, emotional or functional manifestations, or any of the clinical parameters or observations) associated with the disorder, disease, condition or syndrome. As used herein, the terms “treat”, “treatment”, and “treating” also refer to the delay or amelioration or slowing down or prevention of the progression (i.e., the known or expected progression of the disease), severity, and/or duration of the disease or delay or amelioration or slowing down or prevention of the progression of one or more clinical parameters associated with the disease (i.e., “managing” without “curing” the condition), resulting from the administration of one or more therapies.

Treating cognitive impairment according to the invention may involve improving cognition or improving cognitive function as determined by tools used in the field. It may also involve total or partial reversal of cognitive dysfunction. It may also involve attenuation or stopping the progression of cognitive impairment.

### The NO-sGC-cGMP Pathway in the CNS

In the body, nitric oxide (NO) is synthesized from arginine and oxygen by various nitric oxide synthase (NOS) enzymes and by sequential reduction of inorganic nitrate. Three distinct isoforms of NOS have been identified: inducible NOS (iNOS or NOS II) found in activated macrophage cells; constitutive neuronal NOS (nNOS or NOS I), involved in neurotransmission and long-term potentiation; and constitutive endothelial NOS (eNOS or NOS III), which regulates smooth muscle relaxation and blood pressure.

sGC is the primary receptor enzyme for NO in vivo. sGC can be activated via both NO-dependent and NO-independent mechanisms. In response to this activation, sGC converts guanosine-5′-triphosphate (GTP) into the secondary messenger cyclic guanosine 3′, 5′-monophosphate (cGMP). The increased level of cGMP, in turn, modulates the activity of downstream effectors including protein kinases, phosphodiesterases (PDEs), and ion channels.

Intracellular cGMP activates cGMP-dependent protein kinase (PKG) and other downstream modulators and regulates vascular tone and regional blood flow, fibrosis, and inflammation, and has been implicated in neuronal survival and function (Ben Aissa M, Lee S H, Bennett B M, Thatcher G R. Targeting NO/cGMP Signaling in the CNS for Neurodegeneration and Alzheimer's Disease. Current medicinal chemistry; 2016; 23(24):2770-88). Experimental and clinical evidence has indicated that reduced NO concentrations, reduced NO bioavailability, and/or reduced responsiveness to endogenously produced NO contributes to the development of disease. In particular, impaired NO-sGC-cGMP signaling is believed to play an important role in the pathogenesis of many CNS diseases, including those that affect cognition and functional capacity. Impairment of the NO-sGC-cGMP signaling pathway is associated with the pathogenesis of neurodegenerative diseases and has been observed in vascular dementia, AD, and general cognitive impairment (Bennett S, Grant M M, Aldred S (2009), Oxidative stress in vascular dementia and Alzheimer's disease: a common pathology. J Alzheimers Dis 17:245-257; Stephan B C M, Harrison S L, Keage H A D, Babateen A, Robinson L, Siervo M (2017), Cardiovascular Disease, the Nitric Oxide Pathway and Risk of Cognitive Impairment and Dementia. Curr Cardiol Rep 19:87). NO bioavailability and disrupted NO-sGC-cGMP signaling may be impaired by several mechanisms, including endothelial dysfunction and concomitant reduction in endothelial nitric oxide synthase (eNOS) activity, increased levels of the nitric oxide synthesis (NOS) inhibitor asymmetric dimethyl arginine, and increased oxidative stress and reactive oxygen species that react with NO. Endothelial cell loss and NO dysregulation are recognized as major contributing factors in neurodegenerative diseases, resulting in reduced blood flow, vascular leakage, and inflammation, along with synaptic dysfunction and neuronal loss (Toth P, Tarantini S, Csiszar A, Ungvari Z (2017) Functional vascular contributions to cognitive impairment and dementia: mechanisms and consequences of cerebral autoregulatory dysfunction, endothelial impairment, and neurovascular uncoupling in aging. Am J Physiol Heart Circ Physiol 312:H1-H20).

sGC stimulators are a class of heme-dependent agonists of the sGC enzyme that work synergistically with varying amounts of NO to increase its enzymatic conversion of GTP to cGMP. sGC stimulators are clearly differentiated from and structurally unrelated to another class of NO-independent, heme-independent agonists of sGC known as sGC activators. The benzylindazole compound YC-1 was the first sGC stimulator to be identified. Several sGC stimulators have been identified and pharmacologically characterized since then, including BAY 41-2272, BAY 41-8543, riociguat (BAY 63-2521), vericiguat, olinciguat (IW-1701), and praliciguat (IW-1973). To date, the only FDA-approved sGC stimulator is riociguat, which is indicated for the treatment of pulmonary arterial hypertension (PAH) and chronic thromboembolic pulmonary hypertension (CTEPH). No sGC stimulators have been approved for marketing in the field of CNS to date and, to our knowledge, Compound I is the only CNS-penetrant sGC stimulator currently in development for the treatment of CNS diseases.

sGC stimulators may offer considerable advantages over other potential therapies that target the aberrant NO pathway or otherwise upregulate the NO pathway. For example, sGC stimulation is a more powerful approach than either the use of NO supplementation (which is associated with tachyphylaxis) or inhibition of cGMP breakdown (via phosphodiesterase inhibitors [PDEi]), which has limited effectiveness if cGMP levels are very low. In addition, the broad CNS distribution of sGC enables augmentation of signaling across brain regions, while the PDEi targets have more limited cellular and tissue.

### Compound I: IW-6463

Compound I (IW-6463, IWP-247) is an orally administered central nervous system (CNS)-penetrant sGC stimulator being investigated for the treatment of CNS diseases (NCT03856827, NCT04240158 and NCT04475549). To our knowledge it is the only CNS-penetrant stimulator tested in human subjects to date.

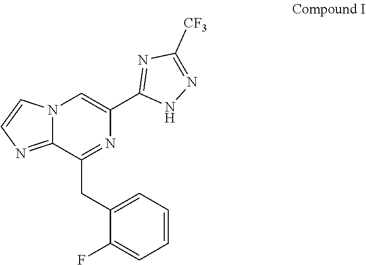 [SMILES: Fc1ccccc1Cc1nc(-c2nc(C(F)(F)F)n[nH]2)cn2ccnc12]

In pre-clinical models, increased concentration of cGMP in the brain, as a result of sGC stimulation by Compound I was shown to lead to increased cerebral blood flow (CBF) and activation of subcortical brain structures relevant to cognition, including some brain regions associated with memory and arousal as assessed by functional magnetic resonance (fMRI-BOLD; see WO 2018/045276 and C. J. WINROW, J. E. JONES, P. GERMANO, S. JACOBSON, S. S. CORREIA, K. W. TANG, J. TOBIN, R. R. IYENGAR, P. P. KULKARNI, C. F. FERRIS, M. CURRIE, J. R. HADCOCK, A central nervous system-penetrant soluble guanylate cyclase stimulator increases cerebral blood flow and modulates fMRI-BOLD responses in rodents. Program No. 692.29. 2018 Neuroscience Meeting Planner. San Diego, CA: Society for Neuroscience, 2018. Online link: https://www.abstractsonline.com/pp8/#!/4649/presentation/2572).

Compound I was also shown to have positive effects on neuroinflammation and displayed neuroprotective properties, in addition to displaying effects on memory and learning (see WO 2018/045276 and S. CORREIA, J. E. JONES, C. REX, G. LIU, A. CARVALHO, P. GERMANO, R. R. IYENGAR, C. J. WINROW, M. G. CURRIE, J. R. HADCOCK, A central nervous system-penetrant soluble guanylate cyclase stimulator reduced spine density loss in aged rats and mice. Program No. 692.26. 2018 Neuroscience Meeting Planner. San Diego, CA: Society for Neuroscience, 2018. Online link: https://www.abstractsonline.com/pp8/#!/4649/presentation/18128; and J. E. JONES, C. J. WINROW, S. S. CORREIA, S. JACOBSON, R. HODGSON, J. PUOLIVALI, K. LEHTIMÄKI, A. CARVALHOA, P. GERMANO, J. V. TOBIN, K. TANG, R. R. IYENGARA, M. G. CURRIE, J. R. HADCOCK, The brain penetrant soluble guanylate cyclase stimulator IWP-247 improved thigmotaxis and increased hippocampal N-acetylaspartate (NAA) concentrations in aged rats. Program No. 692.27. 2018 Neuroscience Meeting Planner. San Diego, CA: Society for Neuroscience, 2018. Online link: https://www.abstractsonline.com/pp8/#!/4649/presentation/18129)

### EEG and Saccadic Eye Movement (SEM) Measurements

Electroencephalography (EEG) is a technique that measures electrical activity in the brain. qEEG stands for quantitative electroencephalography. An event-related potential (ERP) is “a time-locked measure of electrical activity of the cerebral surface representing a distinct phase of cortical processing” for example in response to an auditory or visual stimulus (Patel and Azzam (2005), Characterization of N200 and P300: Selected Studies of the Event-Related Potential. International Journal of Medical Sciences 2(4):147-154). ERPs are time-locked and represent the average of the electrical responses observed after multiple attempts.

EEG-power spectra signals may be analyzed at different frequencies or frequency bands. They were analyzed at the following frequency bands in the experiment described in Example 1: Delta-1-4 Hz (typically associated with sleep), Theta-4-7.5 Hz (associated with waking/falling asleep, some association with cognition), Alpha-8-12 Hz (associated with passive wakefulness, and with cognitive processing), Beta-12-25 Hz (associated with alertness and concentration) and Gamma-25-45 Hz (associated with higher cognitive function).

During EEG experiments based on auditory stimuli such as the ERP measurements described in Example 1, two key waveforms are commonly evaluated following each deviant tone: N200 (associated with stimulus identification and distinction) and P300 (associated with selective attention, information processing and cognitive speed/capacity). In addition, two key parameters are used to quantify each response: latency (how long after the stimulus is the peak signal) and amplitude (how strong is the peak signal).

P300 is a component of the ERP named for its polarity and approximate latency. It is a large positive waveform reaching a maximum at ˜300 ms after stimulus.

N200 is a negative waveform at ˜200 ms after stimulus, associated with stimulus identification and distinction. Mismatch negativity (MMN) is an alternative terminology used for N200 abnormal activity on an auditory ERP that occurs when a sequence of repetitive sounds is interrupted by an occasional “oddball” sound that differs in frequency or duration. MMN or N200 is not dependent upon active engagement on the part of the subject. Both terms are used interchangeable throughout this disclosure. The investigation of MMN in monkeys has shown that NMDA antagonists block the generation of the MMN response, suggesting that NMDA receptors play an important role in this index of information processing and working memory. Consequently, MMN is one of a family of EEG signals that may hold promise as a translational biomarker for CNS diseases, as do other markers of neuronal network activity such as gamma-band oscillation that are aberrant in these patients. Network oscillations may be valuable tools in pharmacological and translational studies that are aimed at developing and refining new treatment interventions for CNS diseases.

Shorter P300 and N200 latencies and larger amplitudes are associated with superior information processing.

Abnormal EEG findings have been linked to dementia and AD (see for example: Palop J J, Mucke L, Network abnormalities and interneuron dysfunction in Alzheimer disease. Nat Rev Neurosci. 2016; 17(12):777-792).

Changes in EEG power spectra have been detected in neuropsychiatric and neurodegenerative disorders and have been correlated with cognitive performance (see for example: Herrmann C S, Demiralp T, Human EEG gamma oscillations in neuropsychiatric disorders. Clin Neurophysiol. 2005 December; 116(12):2719-33; K. van der Hiele, A. A. Vein, R. H. A. M. Reijntjes, R. G. J. Westendorp, E. L. E. M. Bollen, M. A. van Buchem, J. G. van Dijk, H. A. M. Middelkoop, EEG correlates in the spectrum of cognitive decline. Clinical Neurophysiology. Volume 118, Issue 9, 2007, Pages 1931-1939).

Meta-analyses have shown that latency and amplitude of P300 and N200 waveforms are impacted in numerous CNS diseases, are associated with disease progression/severity, may be useful in the analysis of cognitive deterioration and treatment response, and change with aging (see for example van Dinteren R, Arns M, Jongsma M L A, Kessels R P, P300 Development across the Lifespan: A Systematic Review and Meta-Analysis. PLoSONE, 2014, 9(2): e8734; Howe A S, Meta-analysis of the endogenous N200 latency event-related potential subcomponent in patients with Alzheimer's disease and mild cognitive impairment. Clin Neurophysiol. 2014 June; 125(6):1145-51; Ishii R, Canuet L, Aoki Y, Hata M, Iwase M, Ikeda S, Nishida K, Ikeda M, Healthy and Pathological Brain Aging: From the Perspective of Oscillations, Functional Connectivity, and Signal Complexity. Neuropsychobiology 2017; 75:151-161; Patel and Azzam (2005), Characterization of N200 and P300: Selected Studies of the Event-Related Potential. International Journal of Medical Sciences 2(4):147-154).

P300 is disrupted in a variety of neuropsychiatric and neurodegenerative disorders associated with cognitive impairment, including AD and schizophrenia and is proposed to reflect attention, cognitive speed and capacity. N200 is also linked to cognitive processes and has been shown to be altered in AD subjects , other neurodegenerative diseases and psychiatric diseases.

Aging has been shown to result in increased latency and decreased amplitude in P300 signals and also to be associated with decreased cognitive performance and decreases in gamma oscillations and alpha band power .

AD has also been associated with increased latency and decreased amplitude in P300 signals and decreased cognitive performance . It has also been associated with increased latency and decreased amplitude in N200 signals and these values have been correlated with disease severity . It has also been associated with increases in theta and delta activity and decreases in alpha and beta activity and decreases in gamma range. Attenuation of theta power and increase in lexical processing task has been observed in MCI subjects who later converted to AD. Both MCI converters and non-converters showed attenuated alpha suppression with word repetition. ERP has also been used extensively to study subjects with schizophrenia and autism.

The ability to normalize altered EEG signals, as measured by P300, N200, or EEG power spectra, indicates potential for a pharmacological approach to provide benefit in disease states.

In pre-clinical models, sGC stimulation by Compound I was also shown to lead to changes of qEEG signals (Meeting abstracts from the 9th International Conference on cGMP: Generators, Effectors and Therapeutic Implications, Journal of Translational Medicine volume 17, Article number: 254 (2019) S 1-02 Evaluating soluble guanylate cyclase stimulation for serious central nervous system diseases). These studies were performed in rats with telemetry devices implanted in the frontal cortical and front-parietal regions of the brain. Rats were dosed with a suspension of Compound I orally, a suspension of a peripherally restricted sGC stimulator orally, or a solution of donepezil by subcutaneous injection. Compound I altered qEEG measurements including increased gamma oscillations while the peripherally restricted sGC stimulator reduced gamma power compared to vehicle dosing. Compound I given to rats orally at 10 mg/kg increased gamma power and the signal was further increased in combination with 1 mg/kg donepezil at 1-2 hours post-dose.

Nonclinical pharmacology and toxicology data supported conducting clinical studies of Compound Ito assess its safety, tolerability, pharmacokinetic (PK), and pharmacodynamics (PD) in humans. In a Phase I clinical trial (NCT03856827) changes were also observed in the brains of healthy subjects aged 18 to 62 by EEG. More specifically, in that study effects of IW-6463 on ERP P300 amplitude were observed, with an increase in amplitude with increasing dose level, as well as responses being modulated by time since dosing. Improvements in alpha power were also observed in the NCT03856827 study at day 14, across all dosage levels tested and compared to placebo.

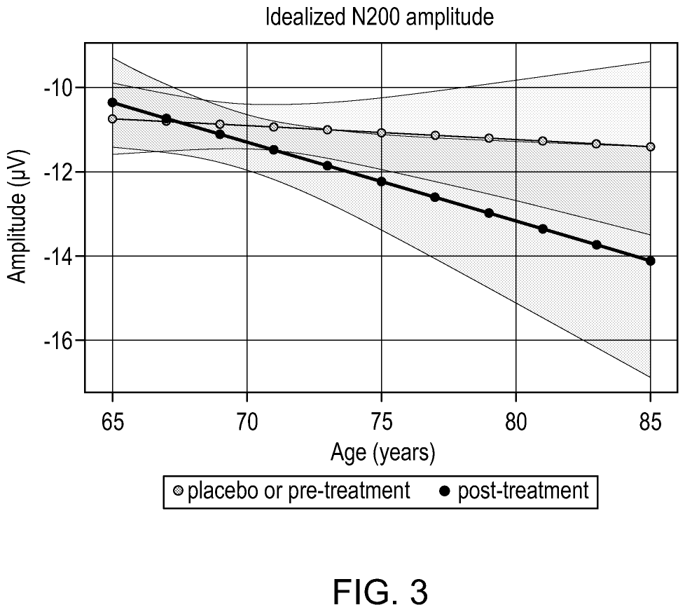

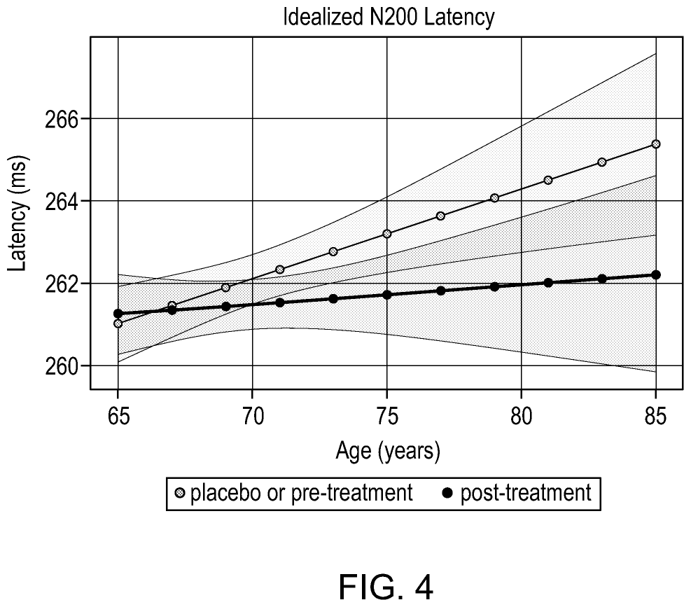

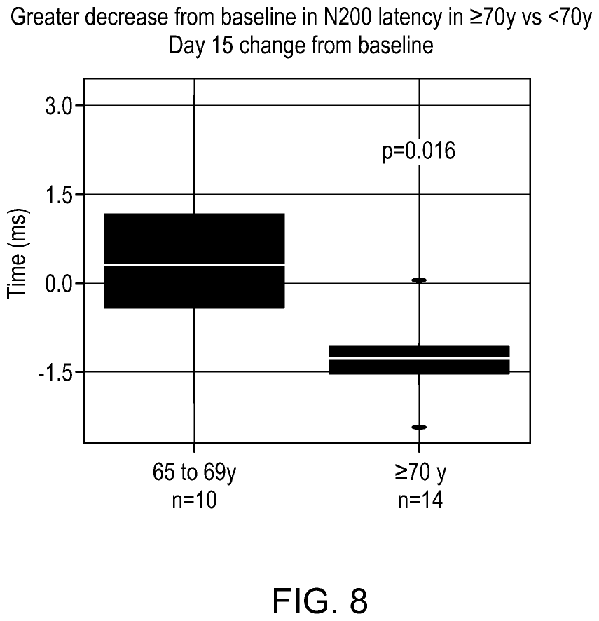

In experiments described in Example 1 section, IW-6463 increased the amplitude (FIG. 3) and decreased the latency of the ERP N200 signal, a neurophysiological biomarker associated with stimulus identification and distinction that is altered in aging, cognitive impairment, and AD. Latency was significantly shorter with IW-6463 treatment at day 14 compared with placebo treatment (p=0.02), an effect that improved with age of participants (FIG. 4 and FIG. 8). Similarly, the effect was also greater in participants who, at baseline, had slower individual alpha frequencies (IAF), a marker of cognitive function/capacity. As seen in FIG. 8, latency response was greater in subjects older than 70 years old as compared with those 65-69 years old (p=0.016). The narrowing of variance for older patients supports a drug effect. At older ages, the magnitude of improvement represents a 10 year age related change in N200 latency after two weeks of treatment with IW-6463.

IW-6463 also increased alpha band power, a parameter known to be decreased in AD and with aging. Alpha band power is also correlated with cognitive decline, APOE4 mutation status, and hippocampal atrophy. A positive impact on EEG (posterior) alpha power, a measure that may reflect attentional processing capabilities, with a 13.7% increase from baseline, i.e. 0.5 dB relative to baseline, in the IW-6463 treatment group compared to a 3.7% decrease (−0.2 dB) in the placebo group (17.4% (0.7 dB) treatment effect, p<0.02) was observed. Trend increases in (anterior) alpha power (17.5% (0.6 dB) in subjects when treated with IW-6463, 4.4% (0.1 dB) in subjects when treated with placebo, 13.1% (0.5 dB) treatment effect; p=0.08) as well as (anterior) gamma power (45% (1 dB) in subjects when treated with IW-6463, −0.1% (−0.3 dB) in subjects when treated with placebo, 44.9% (1.3 dB) treatment effect; p=0.08) were also observed.

Thus, IW-6463 in healthy elderly subjects increased the amplitude and decreased the latency of the ERP N200 signal, a neurophysiological biomarker associated with stimulus identification and distinction that is altered in aging, cognitive impairment, and AD. IW-6463 also increased alpha band power, a parameter known to be decreased in AD and with aging.

A saccade is a short, fast, simultaneous movement of both eyes in the same direction (a jump rather than a smooth movement). Brain areas involved in SEMs include the superior colliculus, substantia nigra, and amygdala. Saccadic peak velocity and latency may be reflective of attention/focus, passive/attentive state, and brain processing time and influenced by factors such as motivation, time on task, and task difficulty. SEMs are very sensitive to sedation, fatigue, and CNS depressants/stimulants and are impacted by aging.

The properties and neurobiology of saccades in both health and disease states have been studied extensively. They have consequently become a valuable diagnostic and research tool (Thurtell, M. J., Tomsak, R. L. & Leigh, R. J, Disorders of saccades. Curr Neurol Neurosci Rep 7, 407-416 (2007)). For example, patients with psychiatric diseases like schizophrenia and depression are known to have increased the latency (time from stimulus to movement) and errors of saccades. Diseases involving musculature (e.g., mitochondrial disease, muscular dystrophy), stroke or head trauma, and neurodegeneration (e.g., Spinocerebral ataxia, Huntington's disease) have also been shown to result in slowing of saccades. The SEMs of AD patients had longer latency and reduced peak velocity when the visual stimulus timing was unpredictable. Aging also decreases peak velocity.

Clinically relevant increases in SEM peak velocity and decreases in latency/reaction time were observed for Compound 1 in the study described in Example 1. Positive effects in this objective SEM performance task related to attention and cognitive processing were observed. These are consistent with improvements observed in other attentional neurophysiological measures discussed above. Saccadic reaction times were significantly shorter (p=0.02) following treatment with IW-6463 compared with placebo treatment. Trends towards increases in saccadic peak velocities were also observed (p=0.07) in response to treatment. The LS mean difference in change from baseline for peak velocity between Compound I treatment and placebo (95% CI) was 28.53 (−2.76, 59.83) p=0.07* (removal of 3-outlier datapoints gave a p value of 0.0391). LS mean difference in change from baseline for latency between Compound I and placebo (95% CI) was −6.58 ms (−11.90, −1.25), p=0.0216.

Some potential future targeted populations being envisioned for treatment with Compound I or a pharmaceutically acceptable salt thereof, among others, include patients with AD, various forms of vascular dementia, or both, including AD with vascular pathology (ADv). ADv patients are those diagnosed with a combination of AD pathology, sub-cortical vascular disease, and cardiovascular risk factors (e.g., hypertension, hypercholesterolemia, elevated BMI and/or diabetes). In some embodiment, ADv patients are AD patients with at least one cardiovascular risk factors. This is a defined subset of the larger AD population, characterized by AD and vascular pathology. Potential targeted populations also include patients suffering from dementia, MCI, subclinical cognitive decline or impairment or SCD due to other diseases, conditions, or events. Potential targeted populations also include patients with mitochondrial diseases and CNS symptoms, such as stroke-like episodes, seizures, migraines, cognitive impairment, aphasia, dysphagia, dysarthria, autism or autism-like features, developmental delays, learning disabilities, vision loss, and movement disorders including ataxia. The treatments here envisioned would also be useful for patients with suboptimal cognitive or functional capacity, for example due to aging.

As mentioned above, cognitive and functional capacity normally deteriorate as a result of aging. It has also been reported that healthy elderly individuals display reduced NO bioavailability compared to younger individuals (Venturelli M, Pedrinolla A, Boscolo Galazzo I, et al. Impact of Nitric Oxide Bioavailability on the Progressive Cerebral and Peripheral Circulatory Impairments During Aging and Alzheimer's Disease. Front Physiol. 2018; 9:169. Published 2018 Mar 14. doi:10.3389/fphys.2018.00169 https://www.frontiersin.org/articles/10.3389/fphyS. 2018.00169/full).

The goal of the study described in the Experimental Section (Example 1) was to assess the effect of a specific dosage of Compound I on a number of parameters or measures related to brain health, cognition, and functional capacity in healthy elderly subjects. The different assessments and measurements carried out are described in detail in the experimental section.

The present invention is based on the surprising findings that a CNS-penetrant sGC stimulator, Compound I, administered at a total oral dosage of 15 mg per day, to a population of healthy elderly subjects in a translational clinical trial, showed evidence of impacts on brain neurophysiology and quantitative brain performance measures in the form of: a) increases in alpha band power and improvements in N200 ERP (MMN) amplitudes and latencies as measured by EEG and b) increases in SEM peak velocity and decreases in latency/reaction time.

### Therapeutic Methods

In some embodiments of the methods and uses of the invention, a therapeutically effective amount of Compound I is a total oral daily dose of between 10 and 15 mg of Compound I. In some embodiments, it is a total oral daily dose of 10 mg. In other embodiments it is a total oral daily dose of 15 mg. In some embodiments, a pharmaceutically acceptable salt of Compound I can be used in the methods and uses of the invention described herein. When a pharmaceutically acceptable salt of Compound I is used, the dose for the pharmaceutically acceptable salt depends on the molecular weight of the salt and has an equal quantity in moles to the dose of Compound I described herein. Accordingly, in some embodiments, the present invention is a method of treating cognitive impairment in a patient in need thereof by administering a total oral daily dose of Compound I of between 10 mg and 15 mg or an equal quantity in moles of a pharmaceutically acceptable salt of Compound I to said patient.

The phrase “pharmaceutically acceptable salt,” as used herein, refers to pharmaceutically acceptable organic or inorganic salts of Compound I. The pharmaceutically acceptable salts of Compound I may be used in medicine. Salts that are not pharmaceutically acceptable may, however, be useful in the preparation of Compound I or of other Compound I pharmaceutically acceptable salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Pharmaceutically acceptable salts of Compound I described herein include those derived from Compound I with inorganic acids, organic acids or bases. In some embodiments, the salts can be prepared in situ during the final isolation and purification of the compounds. In other embodiments the salts can be prepared from the free form of Compound I in a separate synthetic step.

When a compound such as Compound I is acidic or contains a sufficiently acidic moiety, suitable “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N, N′dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When a compound such as Compound I is basic or contains a sufficiently basic moiety, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particular embodiments include citric, hydrobromic, hydrochloric, maleic, phosphoric, sulfuric and tartaric acids. Other exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The preparation of the pharmaceutically acceptable salts described above and other typical pharmaceutically acceptable salts is more fully described by Berg et al., “Pharmaceutical Salts,” J. Pharm. Sci., 1977:66:1-19, incorporated here by reference in its entirety.

In some embodiments of the methods and uses of the invention, treatment with Compound I or a pharmaceutically acceptable salt thereof results in total or partial reversal of cognitive decline. In other embodiments, treatment results in a measurable improvement in cognition in the patient. In other embodiments, treatment with Compound I or a pharmaceutically acceptable salt thereof results in a measurable improvement in cognition in the patient, manifested as improvements in one or more aspects of cognition independently selected from attention, attention span, focus, reaction time to a stimulus, processing speed or combinations of these aspects thereof. In other embodiments, treatment with Compound I or a pharmaceutically acceptable salt thereof results in a measurable improvement in cognition in the patient, manifested as improved memory or improved executive function. In other embodiments, treatment results in preservation in the current level of cognition in the patient. In other embodiments, treatment results in prevention of further deterioration in the level of cognition in the patient (e.g., as compared to the progression that would be expected from what is generally known about the natural progression of aging, the disease, or the condition). In still other embodiments, Compound I is indicated for the treatment of cognitive deficits in a patient in need thereof. In yet other embodiments, treatment is indicated for the treatment of cognitive deficits independently selected from short attention span, distractibility, and lack of focus or combinations thereof.

In some embodiments, treatment with Compound I or a pharmaceutically acceptable salt thereof results in a reduction in neuroinflammation in the patient

In some embodiments of the above methods and uses of the invention, Compound I or a pharmaceutically acceptable salt thereof is indicated for the treatment of cognitive impairment in a patient in need thereof. In some embodiments, it is indicated for the treatment of dementia in a patient in need thereof. In other embodiments, it is indicated for the treatment of MCI in a patient in need thereof. In other embodiments, it is indicated for the treatment of SCD in a patient in need thereof. In still other embodiments, it is indicated for the treatment of sub-clinical cognitive impairment in a patient in need thereof. In yet other embodiments, it indicated for the treatment of cognitive aging.

In some embodiments of the above methods and uses of the invention, treatment with Compound I or a pharmaceutically acceptable salt thereof does not result in an adverse event (AE) associated with symptomatic hypotension.

In some embodiments, treatment with Compound I or a pharmaceutically acceptable salt thereof results in patient's functional capacity.

Improvements in cognitive and functional capacity can be assessed by the improvement or normalization of at least one physiological, physical, psychological, emotional, or any other clinical or pathology parameter associated with cognitive and functional status (e.g., saccadic eye velocity (SEV) or EEG measurements), or improvement of at least one symptom (e.g., short attention span, distractibility, or slow processing speed). In some embodiments, the methods and uses of the invention result in a) increases in alpha band power and/or improvements in MMN (N200) ERP amplitudes and latencies as measured by EEG and/or b) increases in SEM peak velocity and/or decreases in latency/reaction time.

In some embodiments of the methods and uses of the invention, cognitive impairment, either as MCI or dementia, is associated with Alzheimer's disease (AD), vascular dementia, mixed dementia, AD with vascular pathology (ADv), cerebral infarction, cerebral ischemia, stroke, head injury, traumatic head injury, learning disabilities, autism, attention deficit disorder, depression, spinocerebellar ataxia, Lewy body dementia, dementia with frontal lobe degeneration, Pick's syndrome, Parkinson's disease, progressive nuclear palsy, dementia with corticobasal degeneration, amyotrophic lateral sclerosis (ALS), Huntington's disease, demyelination diseases, multiple sclerosis (MS), thalamic degeneration, Creutzfeldt-Jakob dementia, HIV-dementia, schizophrenia, Korsakoff psychosis, post-operative cognitive decline in the elderly, bipolar disorder or mitochondrial disease. In other embodiments, cognitive impairment is associated with sickle cell disease.

In some embodiments of the methods and uses of the invention, the mitochondrial disease is selected from: Alpers Disease, Autosomal Dominant Optic Atrophy (ADOA), Barth Syndrome/LIC (Lethal Infantile Cardiomyopathy), Beta-oxidation defects, Systemic Primary Carnitine Deficiency, Long Chain Fatty Acid Transport Deficiency, Carnitine Palmitoyl Transferase Deficiency, Carnitine/Acylcarnitine Translocase Deficiency, Carnitine Palmitoyl Transferase I (CPT I) Deficiency, Carnitine Palmitoyl Transferase II (CPT II) Deficiency, Very Long-Chain Acyl-CoA Dehydrogenase Deficiency (VLCAD), Long-Chain Acyl-CoA Dehydrogenase Deficiency (LCAD), Long-Chain 3-Hydroxyacyl-CoA Dehydrogenase deficiency (LCHAD), Multiple Acyl-CoA Dehydrogenase Deficiency (MAD/Glutaric acidurioa Type II), Mitochondrial Trifunctional Protein Deficiency, Medium-Chain Acyl-CoA Dehydrogenase (MCAD) Deficiency, Short-Chain Acyl-CoA Dehydrogenase Deficiency (SCAD), Glutaric Aciduria Type II, (SCHAD) Deficiency, Short/Medium-Chain 3-Hydroxyacyl-CoA Dehydrogenase (S/MCHAD), Medium-Chain 3-Ketoacyl-CoA Thiolase Deficiency, 2,4-Dienoyl-CoA Reductase Deficiency, Mitochondrial Enoyl CoA Reductase Protein Associated Neurodegeneration (MEPAN), Carnitine Deficiency, Creatine Deficiency Syndromes, Co-Enzyme Q10 Deficiency, Complex I, II, III, IV, V Deficiency, Chronic Progressive External Ophthalmoplegia (CPEO), Friedreich's Ataxia, Kearns-Sayre syndrome, Leukodystrophy, Leigh Disease or Syndrome, LHON, LHON Plus, Luft Disease, MELAS (Mitochondrial myopathy, encephalomyopathy, lactic acidosis, stroke-like symptoms), Myoclonic Epilepsy with Ragged Red Fibers (MERRF), Mitochondrial Recessive Ataxia Syndrome (MIRAS), Mitochondrial Cytopathy, Mitochondrial DNA Depletion, Mitochondrial Encephalopathy, Mitochondrial Myopathy, Multiple Mitochondrial Dysfunction Syndrome, MNGIE (Myoneurogenic gastrointestinal encephalopathy), NARP (Neuropathy, ataxia, retinitis pigmentosa, and ptosis), Pearson Syndrome, Pyruvate Carboxylase Deficiency, Pyruvate Dehydrogenase Deficiency or Pyruvate Dehydrogenase Complex Deficiency (PDCD/PDH), and POLG Mutations.

In some embodiments of the methods and uses of the invention, MCI, dementia, sub-clinical cognitive impairment, or SCD is associated with cognitive aging, post-operative cognitive decline, medication side-effects, metabolic imbalances, hormonal problems, vitamin or nutrient deficiencies, delirium, psychiatric illness, damage to brain neurons due to an injury (for example in stroke or other cerebral vessel diseases or due to a traumatic brain injury), early stages of a neurodegenerative process, exposure to toxins, or viral or bacterial infections.

In some embodiments of the methods and uses of the present invention, the human patient in need of treatment is one that displays characteristic symptoms or clinical findings associated with cognitive impairment or decline. In other embodiments the patient has been diagnosed as suffering from MCI. In other embodiments, the patient has been diagnosed as suffering from dementia. In other embodiments, the patient has been diagnosed as suffering from vascular dementia, mixed dementia or Alzheimer's disease (AD), or ADv. In other embodiments, the patient has been diagnosed as suffering from a mitochondrial disease. In some embodiments the mitochondrial disease is MELAS. In still other embodiments, the patient suffers from attention deficits or short attention span or distractibility.

In other embodiments of the methods and uses of the invention, the patient displays symptoms or clinical findings associated with sub-clinical cognitive impairment. In some embodiments, the patient is experiencing cognitive aging.

In other embodiments, the patient is one that has not been formally diagnosed with MCI or dementia but he/she or their caretakers report one or more of the characteristic symptoms. In other embodiments, the patient is one that is considered to be at risk of developing cognitive impairment even though he/she has not formally been diagnosed or has not yet developed symptoms.

In still other embodiments, the patient may be one suffering from subjective cognitive decline (SCD).

In some embodiments of the above methods and uses of the invention, the patient is a subject manifesting cognitive impairment, either as MCI or dementia, associated with a disease or disorder selected from AD, vascular dementia, mixed dementia, AD with vascular pathology (Adv), cerebral infarction, cerebral ischemia, stroke, head injury, traumatic head injury, learning disabilities in children, autism, attention deficit disorder, depression, Lewy body dementia, dementia with frontal lobe degeneration, Pick's syndrome, Parkinson's disease, progressive nuclear palsy, spinocereberal ataxia (SCA)dementia with corticobasal degeneration, amyotrophic lateral sclerosis (ALS), Huntington's disease, demyelination diseases, multiple sclerosis (MS), thalamic degeneration, Creutzfeldt-Jakob dementia, HIV-dementia, schizophrenia, bipolar disorder, Korsakoff psychosis, post-operative cognitive decline in the elderly and mitochondrial disease. In other embodiments, the patient is one manifesting cognitive impairment associated with sickle cell disease.

In some embodiments of the methods and uses of the invention, the mitochondrial disease is selected from Alpers Disease, Autosomal Dominant Optic Atrophy (ADOA), Barth Syndrome/LIC (Lethal Infantile Cardiomyopathy), Beta-oxidation defects, Systemic Primary Carnitine Deficiency, Long Chain Fatty Acid Transport Deficiency, Carnitine Palmitoyl Transferase Deficiency, Carnitine/Acylcarnitine Translocase Deficiency, Carnitine Palmitoyl Transferase I (CPT I) Deficiency, Carnitine Palmitoyl Transferase II (CPT II) Deficiency, Very Long-Chain Acyl-CoA Dehydrogenase Deficiency (VLCAD), Long-Chain Acyl-CoA Dehydrogenase Deficiency (LCAD), Long-Chain 3-Hydroxyacyl-CoA Dehydrogenase deficiency (LCHAD), Multiple Acyl-CoA Dehydrogenase Deficiency (MAD/Glutaric acidurioa Type II), Mitochondrial Trifunctional Protein Deficiency, Medium-Chain Acyl-CoA Dehydrogenase (MCAD) Deficiency, Short-Chain Acyl-CoA Dehydrogenase Deficiency (SCAD), Glutaric Aciduria Type II, (SCHAD) Deficiency, Short/Medium-Chain 3-Hydroxyacyl-CoA Dehydrogenase (S/MCHAD), Medium-Chain 3-Ketoacyl-CoA Thiolase Deficiency, 2,4-Dienoyl-CoA Reductase Deficiency, Mitochondrial Enoyl CoA Reductase Protein Associated Neurodegeneration (MEPAN), Carnitine Deficiency, Creatine Deficiency Syndromes, Co-Enzyme Q10 Deficiency, Complex I, II, III, IV, V Deficiency, Chronic Progressive External Ophthalmoplegia (CPEO), Friedreich's Ataxia , Kearns-Sayre syndrome, Leukodystrophy, Leigh Disease or Syndrome, LHON, LHON Plus, Luft Disease, MELAS (Mitochondrial myopathy, encephalomyopathy, lactic acidosis, stroke-like symptoms), Myoclonic Epilepsy with Ragged Red Fibers (MERRF), Mitochondrial Recessive Ataxia Syndrome (MIRAS), Mitochondrial Cytopathy, Mitochondrial DNA Depletion, Mitochondrial Encephalopathy, Mitochondrial Myopathy, Multiple Mitochondrial Dysfunction Syndrome, MNGIE (Myoneurogenic gastrointestinal encephalopathy), NARP (Neuropathy, ataxia, retinitis pigmentosa, and ptosis), Pearson Syndrome, Pyruvate Carboxylase Deficiency, Pyruvate Dehydrogenase Deficiency or Pyruvate Dehydrogenase Complex Deficiency (PDCD/PDH), and POLG Mutations

In some embodiments, the patient is a subject manifesting symptoms of dementia, MCI, subclinical cognitive impairment or SCD that is associated with cognitive aging, post-operative cognitive decline, medication side-effects, metabolic imbalances, hormonal problems, vitamin or nutrient deficiencies, delirium, psychiatric illness, damage to brain neurons due to an injury (for example in stroke or other cerebral vessel diseases or due to a traumatic brain injury), early stages of a neurodegenerative process, exposure to toxins, or viral or bacterial infections.

In some of the above methods and uses, the patient is one that manifests one or more symptoms selected from: deficits in attention, short attention span, distractibility, and slow processing speed.

In some embodiments of the methods and uses of the invention, the human patient is between 65 and 100 years old. In other embodiments, the patient is between 65 and 90 years old. In still other embodiments, the patient is between 65 and 80 years old. In still other embodiments the patient is between 65 and 75 years old. In some embodiments, the human patient is 65 years or older. In other embodiments, the human patient is 75 years or older. In other embodiments, the human patient is 70 years or older. In some embodiments, the human patient is 100 years or older. In still other embodiments, the patient is younger than 65 years old. In still other embodiments, the patient is older than 18 years old. In still other embodiments, the patient is a child. In yet other embodiments, the patient is an adult.

In some embodiments of the methods and uses of the invention, the human patient has an individual alpha frequency (IAF) of less than 10 Hz. In another embodiment, the patient has an IAF of less than 7 Hz. In still other embodiments, of less than 5 Hz.

In certain embodiments, the methods and uses of the present invention described herein comprise administering to the patient a total oral daily dose of between 10 mg and 15 mg. In some embodiments, the methods and uses of the present invention described herein comprise administering to the patient a total oral daily dose of 15 mg. In other embodiments, the methods and uses of the present invention described herein comprise administering to the patient a total oral daily dose of 10 mg.

Even though not tested in the clinical trial described in Example 1, the observed mean plasma and CSF concentrations corresponding to a total oral daily dose of 10 mg (from NCT03856827 trial) also exceed the threshold expected to be needed to achieve some degree of pharmacological activity in the brain, as determined from preclinical models, and is likely to be well tolerated based on clinical results of Phase 1 studies.

In some embodiments of the above methods and uses, the total oral daily dose is given as a single dose (QD). In other embodiments, the total oral daily dose can be split into two equal oral daily dosages (BID) of between 5 mg and 7.5 mg.

In certain embodiments, the methods and uses of the present invention described herein comprise administering to the patient a single oral daily dose of 15 mg of Compound I or an equal quantity in moles of a pharmaceutically acceptable salt of Compound I.

In certain embodiments, the methods and uses of the present invention described herein comprise administering to the patient a single oral daily dose of 10 mg of Compound I or an equal quantity in moles of a pharmaceutically acceptable salt of Compound I.

In certain embodiments, the methods and uses of the present invention described herein comprise administering to the patient two oral daily doses of 5 mg of Compound I or an equal quantity in moles of a pharmaceutically acceptable salt of Compound I.

In certain embodiments, the methods and uses of the present invention described herein comprise administering to the patient two oral daily doses of 7.5 mg of Compound I or an equal quantity in moles of a pharmaceutically acceptable salt of Compound I.

In some embodiments, the methods and uses of the invention described herein comprise administering an initial total oral daily dose of 15 mg of Compound I or an equal quantity in moles of a pharmaceutically acceptable salt of Compound Ito the patient followed by a down-titration to a total oral daily dose of 10 mg of Compound I or an equal quantity in moles of a pharmaceutically acceptable salt of Compound I if the patient does not tolerate 15 mg daily as assessed by a medical practitioner.

In certain embodiments, the methods and uses of the present invention described herein comprise administering to the patient an oral dose of 5 to 7.5 mg of Compound I or an equal quantity in moles of a pharmaceutically acceptable salt of Compound I twice a day. In one embodiment, the methods and uses comprise administering to the patient a first oral dose of 5 to 7.5 mg and a second oral dose of 5 to 7.5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours. In some embodiments, the methods and uses comprise administering to the patient a first oral dose of 5 mg and a second oral dose of 5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours. In other embodiments, the methods and uses comprise administering to the patient a first oral dose of 7.5 mg and a second oral dose of 7.5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In some embodiments, the maintenance dose continues indefinitely as long as the patient continues to experience clinical benefit.

### Combination Therapies

The treatment of cognitive impairment and related symptoms with Compound I or a pharmaceutically acceptable salt thereof can be carried out using the compound alone or in combination therapy with other therapeutic agents. In some embodiments, Compound I or a pharmaceutically acceptable salt thereof can be used for the treatment of cognitive impairment in combination with one or more medications independently selected from cholinesterase inhibitors and NMDA receptor antagonists. In one embodiment, the cholinesterase inhibitor is selected from tacrine, galantamine, donezepil, rivastigmine and combinations thereof. In another embodiment the NMDA antagonist is memantine. In some embodiments, Compound I or a pharmaceutically acceptable salt thereof can be used for the treatment of cognitive impairment in combination with one or more therapeutic agents selected from arginine (e.g., IV or oral), citrulline (e.g., oral), and CoQ10 (e.g., oral).

As used herein, the terms “in combination” (as in the sentence “in combination therapy”) or “co-administration” can be used interchangeably to refer to the use of more than one therapy. The use of the terms does not restrict the order in which therapies are administered to a subject.

The sGC stimulator Compound I or a pharmaceutically acceptable salt thereof can be used in combination therapy with one or more additional therapeutic agents (e.g., additional therapeutic agents described herein). For combination treatment with more than one therapeutic agents where the therapeutic agents are in separate dosage formulations, or dosage forms, the therapeutic agents may be administered separately or in conjunction (i.e., at the same time). In addition, when administered separately, the administration of one therapeutic agent may be prior to or subsequent to the administration of the other agent.

When Compound I or a pharmaceutically acceptable salt thereof is used in combination therapy with other therapeutic agents, a therapeutically effective amount of the other therapeutic agent or each of the other therapeutic agents will depend on the type of drug used. Suitable dosages are known for approved therapeutic agents and can be adjusted by the skilled artisan according to the condition of the subject, the type of condition(s) being treated and the amount of a Compound I or a pharmaceutically acceptable salt thereof being used. In one embodiment of this invention, Compound I or a pharmaceutically acceptable salt thereof, and the additional therapeutic agent are each administered in an therapeutically effective amount (i.e., each in an amount which would be therapeutically effective if administered alone). In other embodiments, Compound I or a pharmaceutically acceptable salt thereof and the additional therapeutic agent are each administered in an amount which alone does not provide a therapeutic effect (a sub-therapeutic dose). In yet another embodiment, Compound I or a pharmaceutically acceptable salt thereof can be administered in a therapeutically effective amount, while the additional therapeutic agent is administered in a sub-therapeutic dose. In still another embodiment, Compound I or a pharmaceutically acceptable salt thereof can be administered in a sub-therapeutic dose, while the additional therapeutic agent is administered in a therapeutically effective amount.

When co-administration involves the separate administration of a first amount of Compound I or a pharmaceutically acceptable salt thereof and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and pharmacokinetic profile. For example, Compound I or a pharmaceutically acceptable salt thereof and the second therapeutic agent can be administered in any order within 24 hours of each other, within 16 hours of each other, within 8 hours of each other, within 4 hours of each other, within 1 hour of each other, within 30 minutes of each other, within 5 minutes of each other, simultaneously or concomitantly.

More, specifically, a first therapy can be administered prior to (e.g., 5 minutes, 15 minutes, 30 minutes, 45 minutes, 1 hour, 2 hours, 4 hours, 6 hours or 12 hours before)), concomitantly with, or subsequent to (e.g., 5 minutes, 15 minutes, 30 minutes, 45 minutes, 1 hour, 2 hours, 4 hours, 6 hours, 12 hours after), the administration of a second therapy to a subject.

### EXAMPLES

For this invention to be better understood, the following examples are set forth. These examples are for purposes of illustration only and are not be construed as limiting the scope of the invention in any manner. All references provided in the Examples are herein incorporated by reference.

### Example 1

Evaluating the Safety, Tolerability, and Pharmacodynamic Properties of IW-6463 in Healthy Elderly Participants

### LIST OF ABBREVIATIONS AND DEFINITION OF TERMS

AD Alzheimer's Disease

AE Adverse Event

ASL Arterial Spin Labeling

AUC Area under the concentration—time curve

BMI Body Mass Index

BOLD Blood Oxygen Level Dependent

BP Blood Pressure

CBF cerebral blood flow

cGMP Cyclic Guanosine Monophosphate

CNS Central Nervous System

CHDR Centre for Human Drug Research

CSF Cerebrospinal Fluid

ECG Electrocardiogram

EEG Electroencephalography

ERP Event Related Potential

FDA Food and Drug Administration

GTP Guanosine Triphosphate

HR Heart Rate

LSM Least Square Means

min Minute(s)

MRI Magnetic Resonance Imaging

NO Nitric Oxide

PD Pharmacodynamic(s)

PK Pharmacokinetic(s)

PLM Passive Limb Movement

SEM Standard Error of the Mean

sGC Soluble Guanylate Cyclase

VAS Visual Analog Scale

### Study Objectives and Outcome Measures

This study was a single-center, double-blinded, randomized, placebo-controlled, two-way cross-over study. Subjects were enrolled into 2 pre-defined, staggered cohorts. The dose selected for each cohort and the start of enrollment within each cohort were based upon the data obtained from another Phase I study (Clinical Trials.gov Identifier NCT03856827) and on the safety profile emerging from that data.

The primary objectives of this interventional clinical study (Clinical Trials.gov Identifier NCT04240158) were: 1) to evaluate the effect of IW-6463 on cerebral blood flow (CBF) in healthy elderly participants by measuring the change from baseline in CBF as measured by magnetic resonance imaging (MRI) and arterial spin labeling (ASL) after administration of IW-6463 vs. placebo; and 2) to assess the safety and tolerability of IW-6463 vs placebo when administered to healthy elderly participants for up to 15 days by determining the number of participants with ≥1 treatment-emergent adverse event (TEAE) after receiving IW-6463 vs placebo.

Exploratory Objectives of this Study were:
1) to evaluate the effect of IW-6463 on systemic vasodilatory properties in healthy elderly participants by measuring the change in blood flow (peak and area under the curve [AUC]) during passive limb movement (PLM) through the femoral artery using Doppler ultrasound imaging after administration of IW-6463 vs. placebo;
2) to evaluate the effect of IW-6463 on functional brain connectivity in healthy elderly participants by measuring the change in functional MRI blood oxygen level-dependent (BOLD) signal after administration of IW-6463 vs. placebo;
3) to evaluate the effect of IW-6463 on brain metabolite levels in healthy elderly participants by determining the change in brain metabolite levels (e.g. glutamate, N-acetylaspartate etc.) as measured by magnetic resonance spectroscopy (MRS) after administration of IW-6463 vs. placebo; the levels of these brain metabolite levels and indicative of the status of cellular energetics in the brain;
4) to evaluate the effect of IW-6463 on cognitive PD assessments in healthy elderly participants by measuring changes in PD parameters over time as assessed by NeuroCart® testing after administration of IW-6463 vs. placebo;
5) to evaluate the effect of IW-6463 on plasma and CSF biomarkers in healthy elderly participants by measuring the change in plasma and CSF biomarkers levels after administration of IW 6463 vs. placebo;
6) to evaluate the PK of daily doses of IW-6463 when administered to healthy elderly participants for up to 15 days by measuring plasma and CSF concentrations of IW-6463 at assessed timepoints; and
7) to evaluate the effect of IW-6463 on alertness, contentedness, and calmness when administered to healthy elderly participants for up to 15 days by obtaining visual analog scale (VAS) measurements at several timepoints during the treatment phase.

### Study Assessments

PLM and the subsequent blood flow increase were measured non-invasively by ultrasound Doppler in the common femoral artery.

Several different functional neuroimaging techniques were used in this trial: MRI-ASL and functional MRI (fMRI). MRI-ASL is used to quantify regional CBF during the resting state. fMRI is a relative measure based on the BOLD effect and it is used to measure the change in CBF in certain regions of the brain as a result of increased brain activity. In addition, the neuronal metabolic profile of the brain (as an indicator of cellular bioenergetics) was measured by magnetic resonance spectroscopy (1H-MRS).

The BOLD effect is based on the fact that blood flow in the brain is highly locally controlled in response to oxygen and carbon dioxide tension of the cortical tissue. When a specific region of the cortex increases its activity in response to a task, the extraction fraction of oxygen from the local capillaries leads to an initial drop in oxygenated hemoglobin (oxyHb) and an increase in local carbon dioxide (CO₂) and deoxygenated hemoglobin (deoxyHb). Following a lag of 2-6 seconds, CBF increases, delivering a surplus of oxygenated hemoglobin, washing away deoxyhemoglobin. It is this large rebound in local tissue oxygenation which is imaged. The reason fMRI is able to detect this change is due to a fundamental difference in the paramagnetic properties of oxyHb and deoxyHb.

NeuroCart® is a full battery of tests for measuring a wide range of CNS functions that was developed by the Center of Human Drug Research (CHDR). NeuroCart can be used to correlate a compound's CNS effects with drug concentration, helping determine whether an effect is due to the compound specifically. NeuroCart provides both objective (e.g. neurophysiology, brain performance) and subjective (e.g. cognitive function, memory, mood, etc.) measures of CNS function. NeuroCart measurements were performed in a quiet room with ambient illumination. Per session, only one participant was allowed in the same room. As used herein, NeuroCart included the following tests:

Saccadic Eye Movements (SEMs)

Saccadic peak velocity is one of the most sensitive parameters for sedation. For instance, while the sedative effects of 20 mg oral temazepam were detectable by participant self-report, visual analogue scales, and SEM testing, at a dose of 5 mg, they were only detectable with measures of SEM. The effects of 1 night of sleep deprivation (suggested as a threshold level of clinically significant sedation) were consistently detectable by SEM testing with a sustained decrease in saccadic peak velocity of 9% to 10% observed.

In this trial, SEMs were recorded at different times. Recording and analysis of SEMs were conducted with a microcomputer-based system for sampling and analysis of eye movements. The program for signal collection and the AD-converter was from Cambridge Electronic Design (CED Ltd., Cambridge, UK), the amplification by Grass (Grass-Telefactor, An Astro-Med, Inc. Product Group, Braintree, MA, USA) and the sampling and analysis scripts were developed at the CHDR (Leiden, the Netherlands). Disposable silver-silver chloride electrodes (Ambu Blue Sensor N) were applied on the forehead and beside the lateral canthi of both eyes of the participant for registration of the electro-oculographic signals. Skin resistance was reduced to less than 5 kOhm before measurements. Head movements were restrained using a fixed head support. The target consisted of a moving dot that was displayed on a computer screen. This screen was fixed at 58 cm in front of the head support. SEMs were recorded for stimulus amplitudes of approximately 15 degrees to either side. Fifteen saccades were recorded with interstimulus intervals varying randomly between 3 and 6 seconds. Average values of latency (reaction time), saccadic peak velocity of all correct saccades and inaccuracy of all saccades were used as parameters. Saccadic inaccuracy was calculated as the absolute value of the difference between the stimulus angle and the corresponding saccade, expressed as a percentage of the stimulus angle. SEMs were recorded in a training session at screening and during each Neurocart session (on day 1 and 15 of each treatment period at 2 hours pre-dose; on day 15 of each treatment period at 2, 3, and 6 hours post-dose). In a quiet room with dimmed lighting, the subject saw a moving dot on a computer screen 58 cm from the head support.

Smooth Pursuit Eye Movements

The same system used for SEMs was also be used for measurement of smooth pursuit. For smooth pursuit eye movements, the target moved at a frequency ranging from 0.3 to 1.1 Hz, by steps of 0.1 Hz. The amplitude of target displacement corresponded to 22.5 degrees eyeball rotation to both sides. Four cycles were recorded for each stimulus frequency.

The time in which the eyes were in smooth pursuit of the target was calculated for each frequency and expressed as a percentage of stimulus duration. The average percentage of smooth pursuit for all stimulus frequencies was used as parameter for analysis.

Body Sway

The body sway meter allows measurement of body movements in a single plane, providing a measure of postural stability. Body sway was measured with a pot string meter (celesco) based on the Wright ataxiameter. At CHDR, the method has been used to demonstrate effects of sleep deprivation, alcohol, benzodiazepines and other psychoactive agents. With a string attached to the waist, all body movements over a period of time were integrated and expressed as mm sway. In this trial, participants were instructed to wear a pair of comfortable, low-heeled shoes on each session. Before starting a measurement, participants were asked to stand still and comfortably, with their feet approximately 10 cm apart and their hands in a relaxed position alongside the body, with their eyes closed. Participants were not permitted to talk during the measurement. The total period of body-sway measurement was 2 minutes.

Adaptive Tracking

The adaptive tracking test was performed as originally described by Borland and Nicholson (Borland, R. G. and A. N. Nicholson, Visual motor co-ordination and dynamic visual acuity. Br J Clin Pharmacol, 1984. 18 Suppl 1(Suppl 1): p. 69s-72s.; Borland, R. G. and A. N. Nicholson, Comparison of the residual effects of two benzodiazepines (nitrazepam and flurazepam hydrochloride) and pentobarbitone sodium on human performance. Br J Clin Pharmacol, 1975. 2(1): p. 9-17), using customized equipment and software (based on TrackerUSB hard-/software (Hobbs, 2004, Hertfordshire, UK)). This test is more sensitive to impairment of eye-hand coordination by drugs than compensatory pursuit tasks or other pursuit tracking tasks, such as the pursuit rotor. The adaptive tracking test has proved to be useful for measurement of CNS effects of alcohol, various other psychoactive drugs, and sleep deprivation.

Adaptive tracking is a pursuit-tracking task in which a circle moves randomly about a screen. The participant must try to keep a dot inside the moving circle by operating a joystick. If this effort is successful, the speed of the moving circle increases. Conversely, the velocity is reduced if the test participant cannot maintain the dot inside the circle.

For this trial, each adaptive tracking test was preceded by three training sessions and included two baseline measurements. After 4 to 6 practice sessions, learning effects were limited. The average performance and the standard deviation of scores over a 3.5-minute period was used for analysis. This 3.5-minute period included a run-in time of 0.5 minutes; data from this run-in time was not recorded.

Visual Verbal Learning Test (VVLT)

In the VVLT, participants were presented with 30 words in 3 consecutive word trials (ie, word learning test VVLT30). Each trial ended with a free recall of the presented words (Immediate Recall, which determines acquisition and consolidation of information). Approximately 30 minutes after start of the first trial, participants were asked to recall as many words as possible (Delayed Recall, which measures active retrieval from long-term memory). Immediately thereafter, the participants underwent a memory recognition test that consisted of 15 presented words and 15 “distractors” (Delayed Recognition, which tests memory storage). Importantly, participants were not allowed to write down words at any time during the test procedure.

N-Back

The N-Back test consists of three conditions, with increased working memory load:
(Condition 0) “X” condition, in which participants were required to indicate whether the presented letter is a “X” (=target) or another letter;
In Condition 1 and 2, letters were presented sequentially (1.5 seconds for a letter [consonant, except for the letter “z”] presentation, followed by a black screen for 0.5 seconds). Key “z” was pressed for a target and “/” was pressed for a non-target.
Condition 1, “1-back” condition, in which participants were required to indicate whether the letter presented was a repetition without any other letter intervening (e.g., B . . . B);
Condition 2, “2-back” condition, in which participants were required to indicate whether a letter was repeated with one other letter in between (e.g., B . . . C . . . B).

The 3 conditions were presented in 3 blocks with increasing working memory load. Each condition started with a training (7 consonants; target:non-target 3:4), followed by the test (24 consonants; target:non-target 1:3).

ECG

ECGs were obtained during the course of the study using Marquette 800/2000/5500 or Dash 3000 and stored using the MUSE Cardiology Information System. ECGs were taken after the participant had rested quietly for at least 5 minutes in the supine position. The investigator assessed the ECG recording as ‘normal’, ‘abnormal—not clinically significant’, or ‘abnormal—clinically significant’ and included a description of the abnormality as required. The ECG parameters assessed included heart rate, PR, QRS, QT, and QTcF (calculated using Fredericia's method).

EEG

Resting-state EEG recordings with open and closed eyes for 5 min in each eye state were performed. Each recording employed alternating periods with eyes open and closed with a duration of 64-seconds for each period. Participants faced a featureless wall and were instructed not to stare, not to move their head and eyes, and to suppress eye blinks.

An EEG cap with electrodes at the Fz, Cz, Pz, and Oz positions (referring to frontal, central, parietal, and occipital regions at the midline, respectively) was used to record signals. Analyses of EEG-power spectra signals were carried out at the following frequency bands:
Delta-1-4 Hz (typically associated with sleep), Theta-4-7.5 Hz (associated with waking/falling asleep, some association with cognition), Alpha-8-12 Hz (associated with passive wakefulness, and with cognitive processing), Beta-12-25 Hz (associated with alertness and concentration) and Gamma-25-45 Hz (associated with higher cognitive function).

In P300 and N200 ERP studies, participants were seated wearing the EEG cap and headphones and were instructed to sit still and relax. During the task, participants were presented with auditory tones. Participants were instructed to pay attention to the tones and press a response-button when they heard an infrequent/deviant tone. A total of 500 tones were presented over 7 minutes, of which 400 were presented as frequent stimuli and 100 as deviant/infrequent stimuli. Therefore, infrequent tones had a probability of 0.2. The first 5 trials were frequent tones and there were at least 2 frequent tones between 2 deviant/infrequent tones. The frequent and infrequent tones were 150 ms tones of 500 Hz and 1000 Hz at a sound pressure level of 75 dB, respectively. All tones has a 5 ms rise and fall time. Tones were presented at a fixed rate of 1 Hz.

VAS Measurements

For the VAS measurements (questionnaires), at certain pre-specified times, the participant indicated (with a mouse click on the computer screen or by drawing a line on a piece of paper) on 16 horizontal visual analogue scales how he/she was feeling. From these measurements, 3 main factors were calculated as described by Bond and Lader (Bond, A. and M. Lader, The use of analogue scales in rating subjective feelings. Psychology and Psychotherapy: Theory, Research and Practice, 1974. 47(3): p. 211-218): alertness (from 9 scores), contentedness (often called mood; from 5 scores), and calmness (from 2 scores).

In addition to the above assessments, vital signs and physical examination as well as blood and cerebro spinal fluid (CSF) sampling were carried out.

Vital Signs

Evaluations of systolic and diastolic blood pressure, pulse rate, respiratory rate, and temperature were performed throughout the study. Pulse and blood pressure were taken after the participant had rested quietly for 5 minutes in supine position. Automated oscillometric blood pressures and pulse rate were measured using a Dash 3000, Dash 4000, Dynamap 400, or Dynamap ProCare 400.

Weight and Height

Weight (kg) was recorded at screening and the follow-up visit or upon early termination. Height (cm) was recorded and body mass index (BMI) calculated at screening.

Physical Examination

Physical examination (i.e., inspection, percussion, palpation, and auscultation) was performed during the course of the study. Clinically relevant findings that were present prior to study drug initiation were recorded with the participant's Medical History. Clinically relevant findings found after study drug initiation and meeting the definition of a TEAE (new AE or worsening of previously existing condition) were recorded.

Laboratory Assessments

Blood and other biological samples were collected for standard clinical laboratory tests, including hematology, chemistry, electrolites, coagulation, glucose, virology, urinalysis, alcohol, and urine drug screenings.

Biomarkers measured in plasma included: asymmetric dimethylarginine (ADMA), symmetrical dimethylarginine (SDMA) and L-arginine (LA), cGMP and biomarkers indicative of NO pathway function; soluble vascular cell adhesion molecule-1 (sVCAM-1) and neurofilament light chain (NF-L), biomarkers of inflammation.

Biomarkers measured in the CSF included: neurofilament light chain (NF-L) and cGMP. Other biomarkers measured in CSF include A2M, C3, TIMP1, PAI1 MCP1, PARC (CCL18), MMP3 and TNFR2.

### Study Design

This single-center double-blind, randomized, placebo-controlled, two-way, cross-over study evaluated a single dose level of IW-6463 compared to placebo. Subjects (male and female) were enrolled into 2 pre-defined, staggered cohorts (Cohort 1 and Cohort 2). The dose selected for both cohorts was the same but the start of enrollment within each cohort was different. This was based upon the availability of preliminary data from the previously started Phase 1 study NCT03856827 in healthy subjects and the emerging safety profile for that study from which a potential different second dose was being considered, but finally was not selected.

Participants received up to a total of 30 daily doses of study drug administered across two 15-day dosing periods (Period 1 and Period 2) separated by an approximate 27-day washout. Participants were randomized to a sequence of receiving IW-6463 for Period 1 and then placebo for Period 2, or vice versa.

The total duration of study participation for each participant could be up to 116 days divided across up to 3 study phases as described below and summarized in the below schematic. All visits were performed on out-patient basis.
Cohort 1
Cohort 2

Study Schematic

A screening phase: up to 42 days before dosing

The screening phase started after full written, verbal, and signed informed consent had been obtained according to CHDR standard operating procedures (SOPs). A full medical screening (medical history, physical examination, 12-lead electrocardiogram (ECG), routine haematology, biochemistry/ electrolytes and urinalysis was be performed to assess a participant's eligibility for this study. Screening had to be performed within 42 days prior to the first dose administration (Day 1).

A treatment phase: consisting of up to two 15-day dosing periods separated by a washout period of 27 days.

The double-blind treatment phase consisted of up to two 15-day dosing periods separated by a washout of 27 days. For dosing period 1, participants were admitted to the clinical research unit (CRU) on the morning of Day 1, randomized, and study drug administration started on Day 1 (there is no Day 0). Participants were discharged from the CRU approximately 8 hours postdose, at the discretion of the Investigator and after all Day 1 assessments had been performed. Participants continued to take daily doses for 14 days; they self-administered their assigned dose at home on non-clinic days and took the daily dose at the clinic during study visit days. After dosing period 1, participants entered the washout period of at least 27 days. Participants in cohort 1 only then entered dosing period 2 when they were admitted to the CRU on the morning of Day 43 for study drug administration. As with dosing period 1, participants were discharged from the CRU approximately 8 hours postdose, and self-administered their assigned daily doses at home on non-clinic days and took the daily doses at the clinic during study visit days. Due to external complications related to the COVID-19 pandemic, participants in Cohort 2 did not enter Period 2 of the study.

A final Follow-up phase: approximately 13 days

For Cohort 2 only, a final follow-up visit where medical assessments took place 13±3 days after the participant's last study drug administration.

### Dosage Regimen and Administration

One dosage regimen was studied in this clinical trial. For subjects receiving IW-6463 during the given dosing period, IW-6463 tablets were administered orally at a 15-mg total once-daily dose for. For subjects receiving placebo during the given dosing period, placebo tablets to match IW-6463 tablets were used. For Cohort 1, the 15 mg total oral daily dose was selected based on emerging data from the Phase 1 study NCT03856827, pre-clinical qEEG results, as well as multispecies PK parameters. For Cohort 2, in addition to the those data. the emerging data from Cohort 1 of this study supported selection of the 15 mg total oral daily dose.

On non-clinic days, participants were instructed to take study drug once per day (QD) at a fixed time, consistent with the time of in-clinic study drug administration. No up or down titration was allowed.

Participants had a validated, custom-designed app installed on either their mobile device or an on an Apple iPad provided by CHDR and the app sent push notifications to the user to take the study drug and to register the time they took the study drug. In addition, the app queried subjects for adverse event information.

### Study Drug

Compound I was administered as one 10 mg and two 2.5 mg oral immediate-release tablet dosage forms. Placebo was administered as multiples of matching placebo tablets. For the manufacture of these tablets, the active ingredient Compound I was combined with excipients (lactose monohydrate, microcrystalline cellulose, croscarmellose sodium and silicon dioxide) and granulated. The granules were then lubricated with magnesium stearate and compressed into round tablets. The core tablets were sub-coated with a film coat of Opadry White II.

### Inclusion Criteria

Each participant had to meet each of the following criteria to be eligible for enrollment in this study:
1. Must have signed an informed consent form (ICF) before any study-specific procedures were performed.
2. Was fluent in the written and spoken language of the local ICF (ie, Dutch).
3. Age is ≥65 years at the day of first dose administration.
4. Patient had to be ambulatory and in good health as defined by the absence of evidence of any clinically relevant active or chronic disease following detailed medical and surgical history review and a complete physical examination including vital signs, 12-lead ECG, hematology, blood chemistry, and urinalysis. The Investigator was tasked with determining whether a particular finding was clinically significant or not. In making this determination, the Investigator considered whether the particular finding could prevent the participant from performing any of the protocol-specified assessments, could represent a condition that would exclude the participant from the study, could represent a safety concern if the participant participated in the study, or could confound the study-specified assessments
5. Had to be able to understand the commitments of the study, to communicate effectively with the Investigator and site staff and agreed to adhere to all study requirements, including the lifestyle restrictions noted below.
6. Had to be able to undergo MRI scanning procedures.
7. Had a body mass index ≥18 and <32 kg/m² at the Screening Visit.
8. Had a supine systolic blood pressure (BP) in the range of 100 to 160 mmHg, inclusive; and supine diastolic BP is in the range of 60 to 95 mmHg, inclusive, at the Screening Visit. Screening BP was the average of 2 measurements obtained with an appropriately sized cuff at 2-minute intervals after the participant had been resting quietly in a supine position for ≥5 minutes. If the average systolic BP was between 150 and 160 mmHg at Screening, confirmation excluding a history of hypertension was obtained. No more than 4 subjects with systolic BP in this range were allowed to be enrolled in each cohort.
9. Had to agree to refrain from making any major lifestyle changes (e.g., start a new diet or change an exercise pattern) from the time of signature of the ICF until after the Follow-up Period.
10. Female participants had to be postmenopausal. A postmenopausal state is defined as no menses for ≥12 consecutive months without an alternative medical cause. A high follicle-stimulating hormone (FSH) level at screening (>40 IU/L or mIU/mL) in the postmenopausal range could be used to confirm a postmenopausal state.
11. Male participants who had not been surgically sterilized by vasectomy (conducted ≥60 days before the Screening Visit or confirmed via sperm analysis) had to agree to use ≥1 of the following effective contraception methods from the date of signing the ICF until 90 days after receiving his final study drug dose: completely abstain from heterosexual intercourse with non-menopausal women—or—if heterosexually active with women of reproductive potential, use, or have their sexual partners use an Intrauterine device—or—a combination of 2 highly effective birth control methods (e.g., condom with spermicide+diaphragm or cervical cap; hormonal contraceptive [e.g., oral or transdermal patch or progesterone implant]+barrier method)—or—maintain a monogamous relationship with a partner who had been surgically sterilized by bilateral oophorectomy, hysterectomy or tubal sterilization.
12. Male participants had to agree to refrain from donating sperm from the Screening Visit through 90 days after the final dose of study drug.

### Exclusion Criteria

A participant who met any of the following criteria was excluded from the study.
1. Having a clinically relevant history of abnormal physical or mental health interfering with the study as determined from the medical history review and the physical examinations obtained during the screening visit and/or at the start of the first study day for each period as judged by the investigator including (but not limited to), neurological, psychiatric, endocrine, cardiovascular (including recent myocardial infarction), respiratory, gastrointestinal, hepatic, renal disorder or presence of narrow-angle glaucoma) psychiatric (including history of clinical depression or suicidal ideation); or neurological disorder.
2. Had a 12-lead ECG at the Screening Visit demonstrating severe bradycardia (heart rate [HR]<40 beats per minute) or average QT interval corrected for HR using Fridericia's formula (QTcF)≥450 msec for men or ≥470 msec for women.
3. Had a family history of short QT syndrome or long QT syndrome.
4. Had a history or clinical evidence of any disease and/or existence of any surgical or medical condition that might interfere with the absorption, distribution, metabolism, or excretion of the study drugs.
5. Had a history of severe allergies, or history of an anaphylactic reaction to prescription or non-prescription drugs or food.
6. Had a positive test for hepatitis B surface antigen (HBsAg), hepatitis B core antigen antibody [anti-HBc], hepatitis C antibody (HCV Ab), or human immunodeficiency virus antibody (HIV Ab) at screening.
7. Had clinically significant hypersensitivity or allergy to any of the inactive ingredients contained in the active or placebo drug products.
8. Had clinically relevant positive urine drug screen (UDS) or alcohol test at screening and/or upon admission to the Clinical Research Unit (CRU) before each dosing period.
9. Had presence or history (within 3 months of screening) of alcohol abuse confirmed by medical history, or daily alcohol consumption exceeding 2 standard drinks per day on average for females or exceeding 3 standard drinks per day on average for males (1 standard drink=10 grams of alcohol), and the inability to refrain from alcohol during the visits until discharge from the CRU (alcohol consumption will be prohibited during study confinement).
10. Had any concurrent disease or condition that could interfere with, or for which the concomitant treatment might interfere with, the conduct of the study or that would, in the opinion of the Investigator, pose an unacceptable risk to the participant in this study.
11. Had received IW-6463 in a prior study, participated in an investigational drug trial in the 3 months prior to administration of the initial dose of study drug or in more than 4 trials in the prior 12 months, is planning to receive another investigational drug at any time during the study, has an active investigational medical device currently implanted, and/or is planning to have an investigational medical device implanted at any time during the study.
12. Had donated or lost of blood in an amount of more than 500 mL within 3 months (males) or 4 months (females) prior to screening and/or donated any plasma within 2 weeks of the Screening Visit.
13. Had elevated levels (ie, >1.5×the upper limit of normal as defined by the laboratory) at the Screening Visit or at Day 1 of alanine aminotransferase, aspartate aminotransferase, or creatinine.
14. Used any nicotine-containing products (eg, cigarettes, e-cigarettes, vape pens, cigars, chewing tobacco, gum, patches) during the 3 months before Day 1.
15. Used an over-the-counter medication during the 7 days before Day 1, with the exception of paracetamol/acetaminophen (<2 g/day), which was allowed until 2 days before Day 1.
16. Based on Investigator judgment, he/she would have been unable to tolerate EEG cap and protocols.

### Study Population/Demographics

Approximately 24 healthy participants (12 in each cohort) aged 65 or older were randomized in the trial. Cohort 1 (12 participants) completed all periods as planned. Due to external complications related to the COVID19 pandemic, Cohort 2 (12 participants) did not enter Period 2 of the study.

A patient was considered to have completed the study after completing all study periods, including the final study discharge visit.

The study population demographics (by race, ethnicity, age, female vs male, etc) can be summarized as follows: of the 24 randomized participants, 21 (87.5%) were white, 2 (8.3%) were of multiple races, and 1 (4.2%) was black; 14 (58.3%) were male and 10 (41.7%) were female, and age of participants ranged from 65 to 79 years with a mean of 70.1 years.

### Concomitant Drugs/Combination Therapy

No prescription medications and OTC medications were permitted within 7 days prior to study drug administrations or less than 5 half-lives (whichever was longer), and throughout the course of the study until study discharge with the exception of paracetamol/acetaminophen (<2 g/day) which was allowed until 2 days before Day 1, and during the study.

In addition, no vitamin, mineral, herbal, and dietary supplements were permitted within 7 days prior to study drug administrations, or less than 5 half-lives (whichever is longer), and during the course of the study until study discharge.

Some exceptions on the use of concomitant medication could be made at discretion of the investigator; the rationale had to be clearly documented by the investigator.

### Lifestyle Restrictions

In-clinic meals were comparable in composition and time of administration.

Participants were instructed not to change their dietary habits during the course of the study.

During the screening and the follow-up visits, participants were required to fast for at least 4 hours (no food or fluids; water is allowed as required).

Throughout both dosing periods, participants were required to fast minimally 8 hours overnight before the in-clinic study dose administration. Water was allowed as desired.

The consumption of any nutrients or supplements known to modulate CYP enzyme activity was prohibited as follows: Grapefruit- or Seville orange-containing products or quinine-containing drinks [tonic water or bitter lemon]) were prohibited from 3 days before dosing until collection of the final PK blood sample

Any vitamin, herbal supplement, or any supplement for the treatment of erectile dysfunction was prohibited during the 14 days before Day 1 of Period 1 through the final Follow-up Visit

The use of (illicit) drugs including cannabis can influence the measurements. Therefore, using ‘illicit drugs’ were not permitted from 1 month before screening and until the final follow-up. Since poppy seeds can cause a positive ‘drugs’ result; those had be avoided. If a positive result occured without an explanation, the participant was not able participate in the study.

Alcohol was not be allowed from at least 24 hours before screening, dosing and each scheduled visit. At other times throughout the study, participants should not consume more than 2 units of alcohol daily on average (1 unit is 10 grams of alcohol). Participants may undergo unscheduled alcohol breath testing at the discretion of the investigator.

Participants were not allowed to have excessive caffeine consumption, defined as >800 mg per day from 7 days prior to the first dose of the study drug. Participants had to abstain from any caffeine-containing products for 24 hours prior to the in-clinic dose administrations. Caffeine quantities were defined as: one cup of coffee contains 100 mg of caffeine; one cup of tea, or one glass of cola, or 1 portion of chocolate (dark: 100 g, milk 200 g) contains approximately 40 mg of caffeine; one bottle of Red Bull contains approximately 80 mg of caffeine.

Participants had to abstain from the use of tobacco-or nicotine-containing products (including e-cigarettes and patches) until the study discharge visit.

Strenuous physical activity (e.g., heavy lifting, weight or fitness training) was not allowed from 48 hours prior to each study day until discharge from the study unit. Light ambulatory activities (e.g., walking at normal pace) was permitted, with the level of activities kept as similar as possible on all days in the study unit.

### Study Assessments/Results

a) Safety

The safety population is defined as all participants who were randomized and received at least 1 dose of study drug. There were no AE reports of syncope, hypotension or other events potentially related to changes in hemodynamics. There were no blood pressure measurements reported below the normal range. Headache was the most commonly reported CNS event, but rates were similar between treatments.

b) Pharmacokinetics

The PK analysis population is defined as all participants who were randomized, received at least 1 dose of study drug, and have at least 1 measurable drug concentration of Compound 1 in collected samples.

The mean concentration of Compound 1 in the CSF of subjects after receiving Compound I was determined to be 77 nM at 5-hrs post last dose. This concentration is 2-3 times the threshold that was expected to be needed to achieve some degree of pharmacological activity in the brain, as determined from preclinical models.

c) Pharmacodynamics

The analysis population for pharmacodynamics is defined as all participants who were randomized, received at least 1 dose of study drug, and have at least 1 post- baseline assessment of the parameter being analyzed.

i) EEG Results

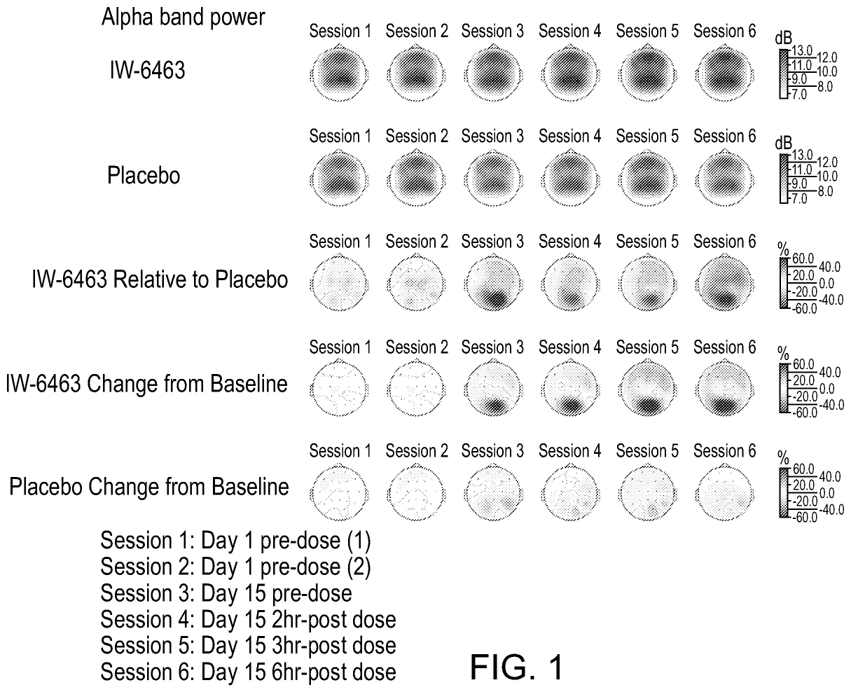

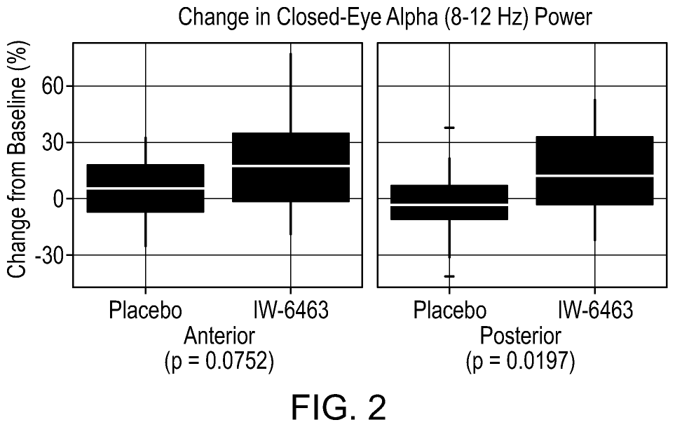

Changes were observed in the alpha frequency range, which is associated with passive wakefulness and with cognitive processing. See FIG. 1 and FIG. 2.

As seen in the FIG. 1, Compound I increased mean alpha band power relative to placebo at all post-baseline timepoints. Effects were observed on Day 15 prior to the last dose as well as 2, 3, and 6 hours following last dose. Increases were observed most prominently in the posterior region though some effects were also observed in the anterior region of the brain.

FIG. 2 summarizes the mean change from baseline in Day 15 alpha band power in anterior and posterior brain regions of subjects when treated with Compound I vs subjects when treated with placebo. Statistically significant impacts on EEG (posterior) alpha power, a measure that may reflect attentional processing capabilities, were observed (13.7% increase from baseline with Compound I treatment compared to a 3.7% decrease with placebo treatment; 17% treatment effect, p=0.02). There were also trend increases in (anterior) alpha power (17.5% increases with Compound I treatment compared to 4.4% with placebo; 13% treatment effect, p=0.08).

Therefore, positive impacts on alpha power, a measure that may reflect attentional processing capabilities, with a significant increase from baseline to day 15 in the Compound I treatment group in posterior alpha compared to the placebo group were observed (p=0.02). These data are supported by trending data on anterior alpha power and gamma power, a measure associated with memory and attention processing.

Improvements in the N200 auditory event-related potential, a measure associated with stimulus identification and distinction were also observed. Latency was significantly shorter with IW-6463 at day 14 compared to untreated subjects (p=0.02).

FIG. 3 is an idealized plot of N200 amplitude vs age. It shows that larger N200 negative amplitudes were observed with increasing age following treatment with Compound I as compared to treatment with placebo or pre-treatment values. This effect was also more pronounced in subjects with slower individual alpha frequencies (IAF) at baseline, a marker of diminished cognitive function/capacity.

FIG. 4 is an idealized plot of N200 latency vs age and shows that smaller latency increases with increasing age were observed following treatment with Compound I as compared to treatment with placebo or pre-treatment values.

FIG. 8 shows mean change versus placebo in N200 latency and how it is driven by the response in older subjects. The latency response was greater in subjects older than 70 years old than in subjects between 65 and 69 years old. The narrowing of variance for older subjects also support a drug effect.

ii) SEM Results

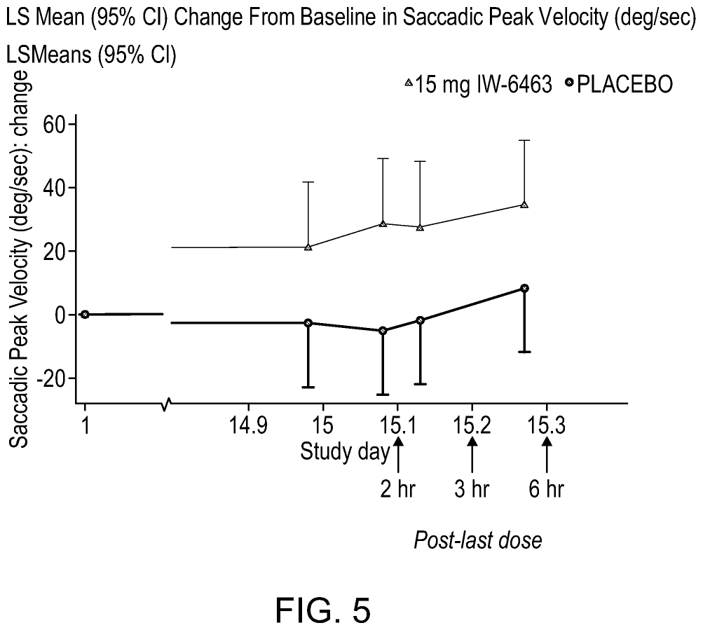

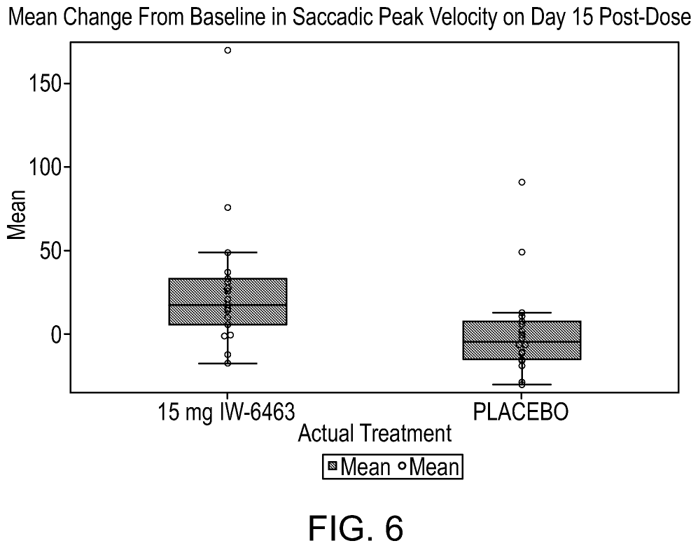

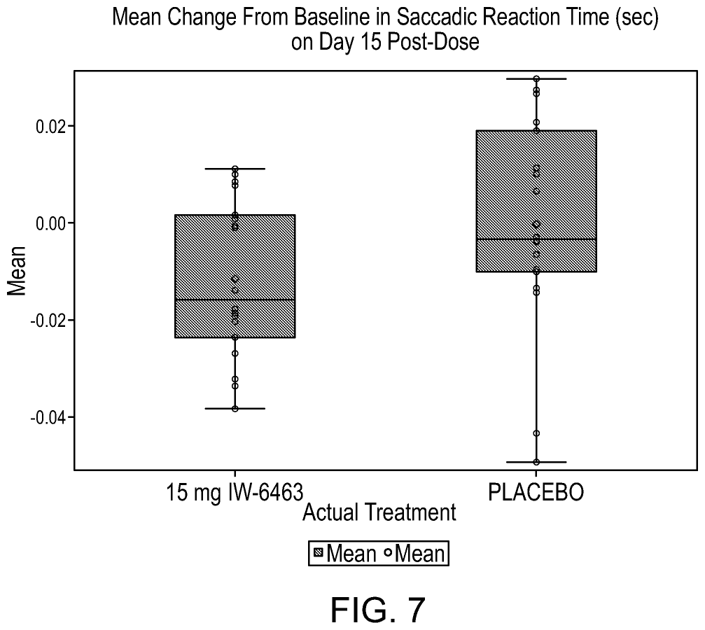

Increases in SEM velocity and decreases in latency/reaction time were observed for Compound I (see FIGS. 5, 6 and 7). The LS mean difference in change from baseline for peak velocity between Compound I treatment and placebo (95% CI) was 28.53 (−2.76, 59.83) p=0.0717* (removal of 3-outlier datapoints gave a p value of 0.0391). LS mean difference in change from baseline for latency between Compound I and placebo (95% CI) was −6.58 ms (−11.90, −1.25), p=0.0216. Thus, positive effects of IW-6463 on an objective SEM task that is related to attention and cognitive processing were observed. Saccadic reaction times were significantly shorter (p=0.02) and there was a trend increase in saccadic velocity (p=0.07). Shorter saccadic reaction times and faster saccadic velocities indicate that IW-6463 is improving CNS functional performance, in the form of motor output, in addition to improving CNS neurophysiology.

iii) Neuroinflammatory Markers

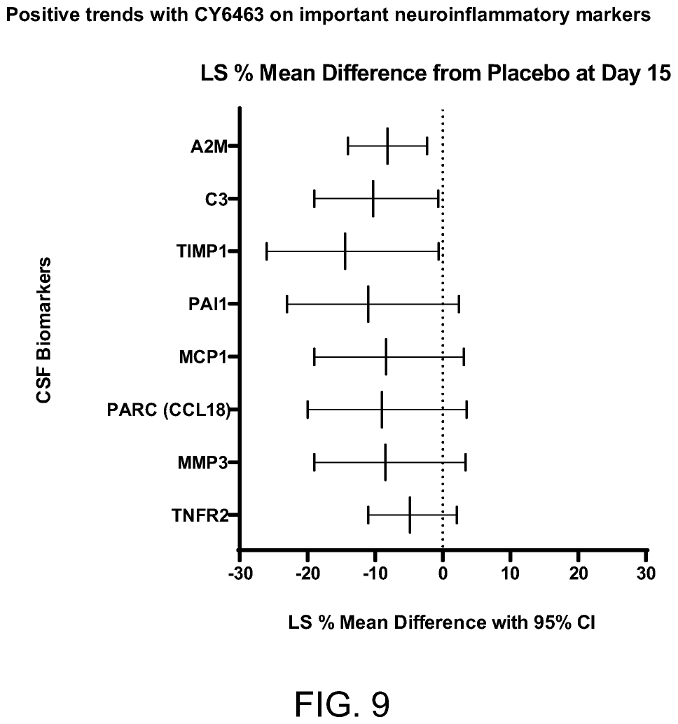

In an exploratory CSF biomarker analysis, positive trends on important CSF neuroinflammatory markers were observed in subjects after two weeks of treatment with IW-6463 as compared with placebo (see FIG. 9, showing results for biomarkers with a nominal p-value cut off of less than 0.2). In particular, potentially important reductions in the concentrations of Alpha 2 macroglobulin (A2M) and complement C3 (C3) biomarkers were observed. The concentration reductions observed for these two biomarkers have 95% confidence intervals that did not cross the zero line, and nominal p values of less than 0.05 (p value is 0.013 for A2M and 0.039 for C3). The larger reductions in concentrations of these biomarkers were observed in subjects older than 70 years old.

A2M elevations are related to cerebrovascular disease and predict cognitive decline and development of AD. They have been reported to lead to tau hyperphosphorylation.

C3 is associated with Aβ and tau and may play a role in synaptic degeneration

1. A method of treating cognitive impairment in a patient in need thereof, by administering to said patient a total oral daily dose of between 10 mg and 15 mg of Compound I:

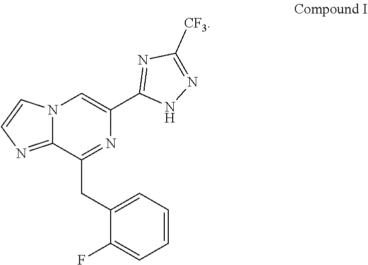 [SMILES: Fc1ccccc1Cc1nc(-c2nc(C(F)(F)F)n[nH]2)cn2ccnc12]

2. The method of claim 1, wherein the cognitive impairment is mild cognitive impairment (MCI), dementia, subclinical cognitive impairment, subjective cognitive decline (SCD) or cognitive aging.

3. The method of claim 1, wherein the cognitive impairment is mild cognitive impairment.

4. The method of claim 1, wherein the cognitive impairment is dementia.

5. The method of any one of claims 1 to 4, wherein the cognitive impairment is manifested as reduced attention, short attention span, distractibility, reduced focus or reduced processing speed.

6. The method of any one of claims 1 to 5, wherein the patient is suffering from Alzheimer's disease (AD), vascular dementia, mixed dementia, AD with vascular pathology (ADv), cerebral infarction, cerebral ischemia, stroke, head injury, traumatic head injury, learning disabilities in children, autism, attention deficit disorder, depression, Lewy body dementia, dementia with frontal lobe degeneration, Pick's syndrome, Parkinson's disease, progressive nuclear palsy, dementia with corticobasal degeneration, amyotrophic lateral sclerosis (ALS), Huntington's disease, cerebrodemyelination diseases, multiple sclerosis (MS), thalamic degeneration, Creutzfeldt-Jakob dementia, HIV-dementia, spinocerebellar ataxia, schizophrenia, Korsakoff psychosis, post-operative cognitive decline in the elderly, bipolar disorder, and mitochondrial disease.

7. The method of any one of claims 1 to 5, wherein the patient is suffering from sickle cell disease.

8. The method of any one of claims 1 to 5, wherein the patient is suffering from Alzheimer's disease (AD), vascular dementia, mixed dementia, or AD with vascular pathology (ADv).

9. The method of any one of claims 1 to 5, wherein the patient is suffering from mitochondrial disease.

10. The method of claim 9, wherein the mitochondria disease is selected from Alpers Disease, Autosomal Dominant Optic Atrophy (ADOA), Barth Syndrome/LIC (Lethal Infantile Cardiomyopathy), Beta-oxidation defects, Systemic Primary Carnitine Deficiency, Long Chain Fatty Acid Transport Deficiency, Carnitine Palmitoyl Transferase Deficiency, Carnitine/Acylcarnitine Translocase Deficiency, Carnitine Palmitoyl Transferase I (CPT I) Deficiency, Carnitine Palmitoyl Transferase II (CPT II) Deficiency, Very Long-Chain Acyl-CoA Dehydrogenase Deficiency (VLCAD), Long-Chain Acyl-CoA Dehydrogenase Deficiency (LCAD), Long-Chain 3-Hydroxyacyl-CoA Dehydrogenase deficiency (LCHAD), Multiple Acyl-CoA Dehydrogenase Deficiency (MAD/Glutaric acidurioa Type II), Mitochondrial Trifunctional Protein Deficiency, Medium-Chain Acyl-CoA Dehydrogenase (MCAD) Deficiency , Short-Chain Acyl-CoA Dehydrogenase Deficiency (SCAD), Glutaric Aciduria Type II, (SCHAD) Deficiency, Short/Medium-Chain 3-Hydroxyacyl-CoA Dehydrogenase (S/MCHAD), Medium-Chain 3-Ketoacyl-CoA Thiolase Deficiency, 2,4-Dienoyl-CoA Reductase Deficiency, Mitochondrial Enoyl CoA Reductase Protein Associated Neurodegeneration (MEPAN), Carnitine Deficiency, Creatine Deficiency Syndromes, Co-Enzyme Q10 Deficiency, Complex I, II, III, IV, V Deficiency, Chronic Progressive External Ophthalmoplegia (CPEO), Friedreich's Ataxia, Kearns-Sayre syndrome, Leukodystrophy, Leigh Disease or Syndrome, LHON, LHON Plus, Luft Disease, MELAS (Mitochondrial myopathy, encephalomyopathy, lactic acidosis, stroke-like symptoms), Myoclonic Epilepsy with Ragged Red Fibers (MERRF), Mitochondrial Recessive Ataxia Syndrome (MIRAS), Mitochondrial Cytopathy, Mitochondrial DNA Depletion, Mitochondrial Encephalopathy, Mitochondrial Myopathy, Multiple Mitochondrial Dysfunction Syndrome, MNGIE (Myoneurogenic gastrointestinal encephalopathy), NARP (Neuropathy, ataxia, retinitis pigmentosa, and ptosis), Pearson Syndrome, Pyruvate Carboxylase Deficiency, Pyruvate Dehydrogenase Deficiency or Pyruvate Dehydrogenase Complex Deficiency (PDCD/PDH), and POLG Mutations.

11. The method of claim 10, wherein the mitochondria disease is MELAS.

12. The method of any one of claims 1 to 11, wherein the patient is between 65 and 100 years old or is older than 100.

13. The method of claim 12, wherein the patient is between 65 and 90, between 65 and 80 or between 65 and 75 years old or older than 70 years old.

14. The method of any one of claims 1 to 13, wherein the patient is administered a total oral daily dose of 15 mg of Compound I.

15. The method of claim 14, wherein the patient is administered a single oral daily dose of 15 mg of Compound I.

16. The method of claim 14, wherein the patient is administered two oral daily doses of 7.5 mg of Compound I.

17. The method of any one of claims 1 to 13, wherein the patient is administered a total oral daily dose of 10 mg of compound I.

18. The method of claim 17, wherein the patient is administered a single oral daily dose of 10 mg of Compound I.

19. The method of claim 17, wherein the patient is administered two oral daily doses of 5 mg of Compound I.

20. The method of claim 16 or 19, wherein the patient is administered a first dose and a second dose, wherein the first dose and the second dose are separated by a period between 5 hours and 15 hours, between 8 hours and 15 hours, or between 10 hour and 15 hours.

21. The method of any one of claims 1 to 20, wherein treatment with Compound I results in a measurable improvement in cognition in the patient.

22. The method of any one of claims 1 to 21, wherein treatment with Compound I results in a reduction in neuroinflammation.

23. The method of claim 21, wherein the improvement in cognition is manifested as improvements in one or more aspects of cognition independently selected from attention, attention span, focus, reaction time to a stimulus, processing speed and a combination of these aspects thereof.

24. The method of claim 21, wherein the improvement in cognition is manifested as improved memory or improved executive function.

25. The method of claim 21, wherein the improvement in cognition is assessed by saccadic eye velocity (SEV) or EEG measurements.

26. The method of any one of claims 1 to 25, wherein the method results in an increase in patient's functional capacity.

27. The method of any one of claims 1 to 25, wherein the method does not result in symptomatic hypotension in the patient.

28. The method of any one of claims 1 to 27, further comprising administering to the patient one or more additional therapeutic agent.

29. The method of claim 28, wherein the additional therapeutic agent is independently selected from cholinesterase inhibitors and NMDA receptor antagonists.

30. The method of claim 29, wherein the cholinesterase inhibitor is selected from tacrine, galantamine, donezepil, rivastigmine and a combination thereof.

31. The method of claim 29, wherein the NMDA antagonist is memantine.

32. The method of claim 28, wherein the additional therapeutic agent is independently selected from arginine, citrulline, and CoQ10.
